(19) United States
(12) Patent Application Publication
(10) Pub. No.: US 2017/0079803 A1
(43) Pub. Date: Mar. 23, 2017

(54) Methods, Devices and Techniques for Improved Placement and Fixation of Shoulder Implant Components

(71) Applicant: ConforMIS, Inc., Bedford, MA (US)

(72) Inventor: Philipp Lang, Lexington, MA (US)

(21) Appl. No.: 15/370,264

(22) Filed: Dec. 6, 2016

Related U.S. Application Data
(62) Division of application No. 14/423,352, filed on Feb. 23, 2015, now abandoned.
(60) Provisional application No. 61/693,748, filed on Aug. 27, 2012.

(51) Int. Cl. A61F 2/40; A61F 2/30; A61B 17/17

(52) U.S. Cl. CPC A61F 2/4081; A61B 17/1778; A61F 2/30942; A61F 2002/30433; A61F 2002/3085; A61F 2002/30878; A61F 2002/30948; A61F 2002/4007; A61B 17/56

(57) ABSTRACT

Improved and/or patient-adapted surgical implants, tools, methods and procedures to assist with the repair and/or replacement of shoulder joints, including the preparation of the glenoid/scapula and/or humeral bones for prosthetic components are disclosed herein.

### RELATED APPLICATIONS

This application is a divisional of U.S. application Ser. No. 14/423,352, entitled “Methods, Devices And Techniques For Improved Placement And Fixation Of Shoulder Implant Components,” filed Feb. 23, 2015, which in turn is a U.S. national stage entry under 35 USC §371 of PCT/US13/56841, entitled “Methods, Devices And Techniques For Improved Placement And Fixation Of Shoulder Implant Components,” filed Aug. 27, 2013, which in turn claims the benefit of U.S. Provisional Application Ser. No. 61/693,748, entitled “Methods, Devices And Techniques For Improved Placement And Fixation Of Shoulder Implant Components,” and filed Aug. 27, 2012. The disclosure of each the above-described applications is hereby incorporated herein by reference in its entirety.

### TECHNICAL FIELD

The disclosure relates to improved and/or patient-adapted (e.g., patient-specific and/or patient-engineered) surgical implants, tools, methods and procedures to assist with the repair and/or replacement of shoulder joints, including the preparation of the glenoid/scapula and/or humeral bones for prosthetic components. More specifically, the disclosure describes systems, tools and methods that facilitate the preparation, implantation and fixation of a glenoid implant component of a shoulder prosthesis.

### BACKGROUND

The natural shoulder joint of an individual may undergo degenerative changes caused by a variety of reasons, including injury, osteoarthritis, rheumatoid arthritis, or post-traumatic arthritis. When such damage or degenerative changes become far advanced and/or irreversible, it may ultimately become necessary to replace all or a portion of the natural shoulder joint with prosthetic shoulder joint components. Shoulder joint replacement, while a relatively recent surgical development over the past few decades, is a well-tolerated surgical procedure that can help relieve pain and restore function in injured and/or severely diseased shoulder joints. Prosthetic shoulder joints are well known in the art, and include a wide variety of different types and shapes of humeral and glenoid components.

In a healthy shoulder joint, the upper end of the humerus typically forms a ball-like structure (the humeral head) which fits into a depression of a socket-like glenoid structure of the scapula. In the traditional implantation of components of a “total-shoulder” prosthesis (e.g., a total shoulder arthroplasty or “TSA implant”), the natural head portion of the humerus is resected and a cavity is created in the intramedullary canal of the patient's natural humerus for accepting a humeral component. The humeral component generally includes a stem and a head portion, which is used to replace the natural head of the humerus. In addition, the glenoid cavity of the scapula may be resected and shaped to accept a glenoid component. The glenoid component generally includes an articulating surface or cup that is secured to the scapula, with a concave surface of the cup facing outwards towards the humeral head, and an opposing surface facing inwards towards the prepared bone surface of the scapula. The glenoid component is desirably engaged by the head portion of the humeral component. Modular designs for the humeral and glenoid components are currently available for the traditional shoulder arthroplasty, and components of different sizes or shapes are at the disposal of the surgeon performing the operation.

A typical glenoid implant component is formed in a relatively circular shape (that substantially matches or follows the natural shape of the glenoid portion of the scapula), with a generally concave joint-facing inner surface and a bone-facing outer surface. The component is intended to fit within a resected portion of the natural glenoid space, with various portions of the natural glenoid material removed during the surgical procedure. In addition to the use of bone cement or other fixation techniques (e.g., impaction, etc.) to fix a glenoid component to the glenoid/scapula, the outer surface of the glenoid component can include one or more short protrusions or tabs that extend into one or more small cavities formed by the surgeon into the neck of the scapula. Because the scapula is a relatively thin bone, however, these protrusions and/or stems are typically limited to a relatively small size and/or shape, and often provide little additional stability to the glenoid component. The lack of available bone for anchoring the glenoid component can be further exacerbated by the presence of significant bone destruction. Despite numerous improvements and advances in the design and placement of shoulder prosthesis components, the malpositioning and loosening of glenoid components remains the primary cause of shoulder joint implant failure. The current revision rates for shoulder arthroplasty are generally accepted at approximately 12%, 15%, and 22%, depending upon the chosen data source as well as the relevant implant components, all of which are much higher than generally accepted revision rates for hip and knee arthroplasty. Accordingly, there is a need for improved methods of positioning, securing and/or anchoring glenoid and/or other implant components within a shoulder joint.

Shoulder hemiarthroplasty is commonly used to treat patients with glenohumeral joint arthrosis. Total shoulder arthroplasty may be indicated for patients without a good articular surface on the glenoid at the time of surgery. For patients with glenohumeral joint arthrosis and an additional deficient rotator cuff, reverse total shoulder arthroplasty may be indicated. One of the leading causes for revision after shoulder arthroplasty results from misalignment of implant components, although there are a number of underlying factors that ultimately contribute to the high revision rate. For example, accurate positioning of the glenoid and humeral cuts and complimentary components is important to achieve a stable joint, and component loosening and instability can often be the result of poor positioning of the component. However, current humeral instrumentation design limits alignment of the humeral resection to average values for inclination and version. While some instrumentation designs allow for adjustability of inclination and offset, assessment is still made qualitatively. Also, surgeons often use visual landmarks, or “rules of thumb,” which can be misleading due to anatomical variability. Similar problems exist with glenoid preparation.

Another problem arising in shoulder arthroplasty is that surgeons often experience difficulties with resurfacing the glenoid due to a lack of exposure. Exposure in shoulder arthroplasty is limited due to the extensive amount of soft tissue surrounding the shoulder compartment. Because of this problem, surgeons may be able to perform only a hemiarthroplasty in which only the humeral head is replaced.

Yet another problem unique to shoulder arthroplasty is the difficulty in determining the thickness of the scapula. Such a determination is necessary to prevent breakthrough during preparation of the glenoid.

In fracture situations, it is difficult to determine the inferior/superior position of the humeral head due to the absence of landmarks. Malpositioning of the humeral head can lead to instability of the shoulder and even dislocation. The surgeon also relies on instrumentation to predict the appropriate size for the humerus and the glenoid instead of the ability to preoperatively and/or intraoperatively template the appropriate size of the implants for optimal performance.

Another challenge for surgeons is soft tissue balancing after the implants have been positioned. Releasing some of the soft tissue attachment points can change the balance of the shoulder; however, the multiple options can be confusing for many surgeons. Moreover, in revision shoulder arthroplasty, many of the visual landmarks may no longer be present, making alignment and restoration of the joint line difficult if not impossible.

### BRIEF DESCRIPTION OF THE SEVERAL VIEWS OF THE DRAWINGS

FIG. 1 depicts a humerus and scapula of an exemplary shoulder joint illustrated schematically to indicate various features and landmark;

FIG. 2 depicts a partial front view of the scapula of FIG. 1;

FIG. 3 depicts a side view of the scapula of FIG. 1;

FIG. 4 depicts an exemplary 3-dimensional wire frame drawing of a scapula;

FIG. 5 depicts one exemplary glenoid canal having been modeled using anatomical image of the scapula of FIG. 4;

FIG. 6 depicts a medial view of exemplary embodiments of a glenoid implant component and associated scapular anchor or stem constructed in accordance with various teachings of the present disclosure;

FIG. 7 depicts a side view of the embodiments of the glenoid implant component and associated scapular anchor of FIG. 6;

FIG. 8 depicts a side view of the glenoid component and scapular anchor of FIGS. 6 and 7, with the anchor docked with and secured to the glenoid component;

FIG. 9 depicts a partial side view of a human torso, with various subcutaneous layers exposed, and a shoulder region being accessed through external skin and soft tissue layers;

FIG. 10 depicts a partial front view of a shoulder and associated soft tissues, and relevant portions of the shoulder region being accessed;

FIG. 11 depicts a partial front of the shoulder of FIGS. 8 and 9, with various tissues retracted and/or exposed;

FIG. 12 depicts side and front views of a guide tool designed using patient-specific image data to include a surface that matches or substantially conforms to a surface of the humerus;

FIG. 13 depicts the tool of FIG. 12, in contact with a humerus;

FIG. 14 depicts a partial front view of a shoulder joint incision including a resected humeral head and prepared humeral intramedullary canal, and a partial cross-sectional view of the glenoid cavity and portions of the scapula;

FIG. 15 depicts a partial view of a scapula with a canal or channel created within a relevant scapular section;

FIG. 16A depicts a normal humeral head and upper humerus which forms part of a shoulder joint;

FIG. 16B depicts a humeral head having an alignment jig designed to identify and locate various portions of the humeral anatomy;

FIG. 16C depicts an alternative embodiment of a humeral head jig that utilizes an alternative conforming surface to align the jig;

FIG. 17A depicts a humeral head with osteophytes;

FIGS. 17B and 17C depict the humeral head of FIG. 17A with a more normalized surface that has been corrected by virtual removal of the osteophytes;

FIG. 18A depicts a humeral head with voids, fissures or cysts;

FIGS. 18B and 18C depict the humeral head of FIG. 18A with a more normalized surface that has been corrected by virtual removal of the voids, fissures or cysts;

FIG. 19A depicts a healthy scapula of a shoulder joint;

FIG. 19B depicts a normal glenoid component of a shoulder joint;

FIG. 19C depicts an alignment jig for use with the glenoid of FIG. 19B;

FIG. 19D depicts a milling and/or reaming operation of the glenoid of FIG. 19C;

FIG. 20A depicts a glenoid component with osteophytes;

FIG. 20B depicts the glenoid component of FIG. 20A with a more normalized surface that has been corrected by virtual removal of the osteophytes;

FIGS. 20C and 20D depict two alternative embodiments of a glenoid jig for use with the glenoid of FIG. 20A, each of which incorporates conforming surfaces that accommodate the osteophytes;

FIG. 21A depicts a glenoid component with voids, fissures or cysts;

FIG. 21B depicts the glenoid component of FIG. 21A with a more normalized surface that has been corrected by virtual “filling” of the voids, fissures or cysts;

FIG. 21C depicts an embodiment of a glenoid jig for use with the glenoid component of FIG. 21A, which incorporates various conforming surfaces that accommodate the voids, fissures and/or cysts (and other surfaces) of the glenoid component; and

FIG. 22 depicts an exemplary flowchart a process beginning with the collection of patient data in process step.

### DETAILED DESCRIPTION

The following description of various embodiments of the disclosure are merely exemplary in nature and are in no way intended to limit the disclosure, its various applications and/or uses. Further areas of applicability of the present teachings will become apparent from the description provided hereinafter. It should be understood that the description and various examples, while indicating various embodiments, are intended for purposes of illustration only and are not intended to limit the scope of the teachings.

The human shoulder joint is primarily made up of three bones, the humerus (or upper arm bone), the scapula (or shoulder blade), and the clavicle (or collarbone), as well as associated muscles, ligaments, tendons and related structures. There are various articulations between the bones of the shoulder, but the major articulation between the humerus and the scapula, or glenohumeral joint, is most commonly referred to as the “shoulder joint.” In humans, articulation of the glenohumeral joint occurs where the humeral head rotates against and sits within the glenoid fossa of the scapula.

In general, there are two kinds of cartilage in the shoulder joint. The first type is articular cartilage on the ends of the bones, which allows the bones to smoothly move over and/or against each other. The second type of cartilage is the labrum, which is a substantially more fibrous and rigid cartilage, found only on the glenoid socket (the rim of the glenoid fossa), and which serves to deepen the glenoid socket so that the humeral head is retained with the glenoid socket during shoulder movement. The labrum also functions as an attachment point for various structures or tissues around the joint, including various ligaments that hold the joint together.

The shoulder is one of the most mobile joints in the human body, and is capable of a remarkable range of abduction, adduction and rotation, as well as the ability to raise the arm in both anterior and posterior directions and move through a full 360 degrees in the sagittal plane. This tremendous range of motion allowed by its construction comes with a price—the incredible mobility renders the shoulder joint extremely unstable, and far more prone to dislocation and/or injury than other joints of the body. Because much of the shoulder joint's motion (and/or various motion limits) is controlled by the numerous soft and connective tissues surrounding the joint, rather than primarily by articulating bony structures such as in the knee and/or elbow, even minor damage to such soft-tissue structures can significantly affect and/or permanently degrade the proper functioning of the joint.

Where disease, injury or other joint defects render a shoulder joint unusable and/or excessively painful, it may be desirous to repair and/or replace some portion or all of the various articulating structures (and/or supporting soft and/or hard anatomical structures) of the joint. For example, the articulation of the humerus with the glenoid (the glenohumeral or shoulder joint) may deteriorate. The humeral head or the glenoid may deteriorate and become rough or lose their anatomical shapes and reduce motion, increase pain, or the like. The labrum of the glenoid may thin, recede, tear, spilt or otherwise deteriorate. These changes may happen for various reasons, such as injury, disease, or lack of motion. This may lead to replacement of the selected portions of the anatomy with a prosthesis to achieve a substantially normal or anatomical range of motion. In many cases, the precise restoration of glenoid orientation using an implant component can be complicated by the very tissue or bone loss and/or destruction that may be responsible, at least in part, for the need for shoulder replacement surgery.

In the past, a shoulder joint exhibiting osteoarthritis or other significant damage and/or degradation could be repaired and/or replaced using standard off-the-shelf implants and other surgical devices. Such implants, which typically employed a one-size-fits-all (or a few-sizes-fit-all) approach to implant design, often resulted in significant differences between a patient's existing or healthy biological structures and the resulting implant component features in the patient's shoulder joint. While other joints may tolerate (to varying degrees) significant disparities between the available and optimal implant sizes and/or shapes, the shoulder can be much less forgiving—a suboptimal size/shaped and/or improperly placed implant component can easily result in a non-functional, unsteady and/or unacceptably painful shoulder joint. This dissimilarity in sizes and/or shapes between the natural anatomy and standard implant components can be further exacerbated by the very “unstable” design and nature of the human shoulder joint. Accordingly, it is highly desirable for the biometrics and/or kinematics of the shoulder to be accurately reconstructed during the surgical procedure.

Moreover, malpositioning of one or more prosthesis components (or component loosening that may eventually alter the prosthesis dynamics) can result in excessive and unacceptable anteversion and/or retroversion of the glenoid components, as can malpositioning of one or more prosthesis components that prevent loading (e.g., eccentric loading) of the glenoid component in a desirable manner. Despite the numerous advances in the designs of glenoid components and the methods and tools used for their installation, such prostheses still lack the stability and strength of natural healthy glenoid components, and the relative orientations and placements of prosthetic glenoid and humeral components most often do not provide proper soft tissue balance.

Moreover, simply gaining access during the surgical repair of a shoulder joint can often be particularly challenging, as the joint is completely surrounded by a joint capsule, and numerous soft and connective tissues are positioned and/or secured on almost every side of the joint. Although it is known in shoulder procedures to replace various portions of the anatomy, such as a humeral head and/or a glenoid, many procedures generally require relatively large incisions through soft tissue. Further, various procedures require that many muscle and muscle attachments (as well as other soft connective tissues) be cut, resected, retracted and/or otherwise manipulated or modified to achieve access to selected portions of the anatomy. Although it may be selected or necessary to perform many procedures in this manner, it may also be desirable to achieve a surgical correction via a less invasive procedure. Where a complete exposure and/or substantial/complete glenohumeral dislocation is undesirable or contraindicated, such as where damage and/or the removal of such tissues can unacceptably destabilize the shoulder joint, the surgical access may be extremely limited (e.g., using less invasive and/or minimally invasive approaches), which can significantly reduce the ability of the surgeon to access and/or directly visualize various anatomical structures within the joint. Such inability to properly visualize and prepare the various anatomical structures, as well as the limited ability to visualize and/or position implant components implanted therein, can significantly reduce the effectiveness of even the most skilled surgeon and surgical repair.

In additional to retention of soft tissues surrounding the joint, another significant factor lending to the success (or contributing to the failure) of total shoulder arthroplasty is the quality of bone stock available for fixation of the implant components. In most cases where there is inadequate bone stock, the deficiency is on the glenoid side. However, even where sufficient bone stock is available, the proper and adequate anchoring of any glenoid components can be challenging, owing at least in part to the small size, unique shape(s) and limited thickness of the healthy scapula. In fact, in long term studies, glenoid loosening is more common than humeral loosening, and glenoid loosening has been found to be the most common long-term complication of total shoulder replacement.

Various embodiments of the present disclosure include the use of patient-specific and/or patient-adapted image data (as well as the possible comparisons with and/or modifications using databases of “normal” or other patient anatomical characteristics) to determine various structural and strength features of anatomical structures, including the humerus and glenoid/scapula of the shoulder. For example, the bone modulus of a scapula can be characterized from the Hounsfield unit measurements obtained from CT scans. Bone with higher modulus is often stronger, and can be an ideal location for the placement of scapular anchors and/or peg/screw fixation, as well as directly or indirectly supporting the various implant components. The surgeon or design engineer can use this localized information to pre-operatively design the various anchors, stems and implant components, as well as corresponding guided tool instruments that prepare and direct the various surgical tools and/or implants into bone of higher modulus (or, if desired, to avoid such bone or other structures when the removal, modification and/or augmentation of weaker bone is desired and/or indicated for various reasons).

Unlike the bony structures available to support hip or knee implants (which may rely on an intramedullary canal of associated long bones for fixation and/or alignment of implant components), the scapula of the shoulder is not technically a “long bone” in the traditional sense, and thus surgeons have not primarily relied upon or employed, in various cases, any clearly defined and readily available intramedullary canal in the scapula for supporting a glenoid component. Moreover, unlike hip or knee replacements (which rely heavily on the intramedullary canal for alignment), surgeons have heretofore relied upon palpation and experience to evaluate and/or determine the anteroposterior and superoinferior tilt of a glenoid component.

The present disclosure includes the use of patient specific data and patient-adapted modeling during the planning and performance of total shoulder arthroplasty (TSA) procedures. Various embodiments include the use of patient data and/or modeling in the creation of patient-specific tools and procedures for preparing the various anatomical surfaces and/or structures of the shoulder joint (e.g., the humerus and/or the scapula) for surgical repair and/or replacement using a variety of surgical tools and implant components. Various embodiments include the use of patient data and/or modeling in the design, selection and/or modification of individual implant components and/or portions thereof, including components used for both joint replacement and/or resurfacing. Moreover, various embodiments include the use of patient data and/or modeling in the design, selection and/or modification of anchoring devices and/or securement strategies for ensuring the adequate and continued fixation of implant components within and/or in relation to designated anatomical structures. In addition, various embodiments include the employment of patient data and/or modeling in the design, selection and/or modification of surgical access procedures or techniques to facilitate access to and preparation of relevant anatomical structures of the shoulder (e.g., bones and/or articular surfaces) in a surgically acceptable manner, which may include the minimal disruption of critical or important soft tissue structures (if desired).

Various embodiments described herein further include the use of patient-specific anatomical data in design and/or selecting of surgical instruments and guide tools for preparing a patient's glenoid and humerus, with the glenoid instrument and companion humeral instruments (e.g., instruments and guide tools) generated and provided to guide and accomplish the resection of bone in preparation for the implantation of the various components of the total shoulder implant system. The various humeral and glenoid instruments can be defined and manufactured from any biocompatible material, including, sterilizable plastic, polymers, ceramics, metals or combinations thereof, using various manufacturing processes. The tools can be disposable and can be combined or used with reusable and non patient-specific cutting and guiding components. The instruments will desirably be steam sterilizable and biocompatible. Both the glenoid and humeral guide tools will desirably include a minimal profile and/or volume, and simulation of passage of these instruments through the chosen incision should be performed prior to manufacture, as the surgical exposure for these types of procedures can be quite small. In various embodiments, the design and/or selection of the various instruments and/or implants may be particularized for an intended resection type and/or direction, such as particularized to allow handle or other feature extension through and/or out of a less-invasive incision and/or designing a guide tool to conform to surfaces directly accessible through one or more pre-specified and/or desired anterior and/or superior incision(s) in the shoulder.

A wide variety of imaging techniques, including Computerized Axial Tomography/Computed Tomography (CAT/CT) scans, Magnetic Resonance Imaging (MRI), and other known imaging techniques, can be used to obtain patient-specific anatomical information. In various embodiments, the patient-specific data can be utilized directly to determine the desired dimensions of the various humeral and scapular/glenoid prosthesis components for use in the total shoulder arthroplasty procedure for a particular patient. Various alternative embodiments contemplate the use of computerized modeling of patient-specific data, including the use of kinematic modeling and/or non-patient data sources, as well as general engineering techniques, to derive desired dimensions of the various humeral and scapular/glenoid prostheses, surgical tools and techniques.

In various embodiments, images or scans of the shoulder area, optionally with scans of the neck and/or elbow, can be used to determine the locations, length and cross-sectional dimensions of the humerus and the scapula, as well as those of the glenoid and the scapular canal(s) or other anatomical features. This data can be used to derive the relationships between the longitudinal axis of a relevant scapular canal and the orientation of the glenoid, including the angles between the canal and the glenoid planes as well as the location of their intersection. Based on the foregoing dimensions and relationships, appropriate features of the glenoid prosthesis, the scapular anchor and the connecting mechanism therebetween can be derived, selected and/or modified such that the glenoid component can fit securely against a prepared scapula/glenoid pocket, and including a desired length, diameter and various tapers of the scapular anchor, the dimensions of the tray and the location and angle of the scapular anchor relative to the glenoid tray. Similar approaches can be utilized for humeral implant components as well.

In various embodiments, patient-specific surgical instruments can include, for example, alignment guides, drill guides, templates, cutting/resection guides for use in shoulder joint replacement, shoulder resurfacing procedures and other procedures related to the shoulder joint or the various bones of the shoulder joint. The patient-specific instruments can be used either with conventional implant components or with patient-specific implant components that are prepared using computer-assisted image methods. The patient-specific instruments and any associated patient-specific implants can be generally designed and formed using computer modeling based on the patient's 3-D anatomic image generated from image scans including, X-rays, MRI, CT, ultrasound or other scans. The patient-specific instruments can have a three-dimensional engagement surface that is complementary and made to conformingly contact and match at only one position a three-dimensional image of the patient's bone surface (which can be imaged selectively with associated soft tissues or without soft tissue, e.g., an actual bone surface), by various methods. The patient-specific instruments can include custom-made guiding formations, such as, for example, guiding bores or cannulated guiding posts or cannulated guiding extensions or receptacles that can be used for supporting or guiding other instruments, such as drill guides, reamers, cutters, cutting guides and cutting blocks or for inserting pins or other fasteners according to a pre-operative plan.

In one exemplary embodiment, patient specific data and patient-adapted modeling can be used to ensure the proper alignment of implant component features relative to the native bones. Once sufficient data is obtained and/or modeled, a designer and/or physician can review the anatomical data and position and size of implant components customized (or to be customized) for the patient. With an estimated implant location and size determined or estimated, Creo Elements/Pro (an image and design processing program commercially available from Parametric Technology Corporation of Needham, Mass., USA) or an equivalent computer program can be used to create a template instrument that can be used to help prepare anatomical structures and/or align the glenoid or other component(s) during the surgery. A portion of the glenoid component instrument can be designed to conform to the native bone. A matching/conforming portion of the instrument can include a surface that is the 3D inverse (or approximation thereof, including a “filtered” approximation) of the native surface of the glenoid created via a Boolean subtraction operation where the native surface of the glenoid is subtracted from the template instrument. An approximately 1 mm gap (or other distance) between the bony surface of the glenoid and the inverse surface of the glenoid component instrument can be added when using CT data to accommodate cartilage and/or slight errors in the reconstruction. The surface can be created using Geomagic software (a computing program commercially available from Geomagic USA of Morrisville, N.C., USA) or equivalent software. Various additional features of the surface can include bony surface features or other structures (e.g., voids, osteophytes and/or other soft and/or hard tissue features) close to but outside of the glenoid articular surface, which can be used to provide further positioning of the instrument with respect to the bone. If desired, the surface may “wrap around” or otherwise encompass some portion of the anterior aspect of the glenoid surface, which may be easier to reference using a traditional delto-pectoral surgical approach. This feature may also be used to “lever” or otherwise position the instrument over and/or around the glenoid. A variety of such features, which can include one, two, three or more such features around the perimeter of the glenoid, can be included in the instrument, depending upon the condition of the bone structure, its geometry, and the relevant surgical exposure.

TABLE 1
Exemplary implant features that can be patient-
adapted based on patient-specific measurements

| Category | Exemplary feature |
|---|---|
| Shoulder implant or guide | One or more portions of, or all of, an external implant |
| tool component | component curvature |
| | One or more portions of, or all of, an internal implant |
| | dimension |
| | One or more portions of, or all of, an internal or external |
| | implant angle |
| | Portions or all of one or more of the ML, AP, SI dimension |
| | of the internal and external component and component features |
| | An locking mechanism dimension between a plastic or |
| | non-metallic insert and a metal backing component in one or more |
| | dimensions |
| | Component height |
| | Component profile |
| | Component 2D or 3D shape |
| | Component volume |
| | Composite implant height |
| | Component articular surface curvature |
| | Component bone-facing surface curvature |
| | Insert width |
| | Insert shape |
| | Insert length |
| | Insert height |
| | Insert profile |
| | Insert curvature |
| | Insert angle |
| | Distance between two curvatures or concavities |
| | Polyethylene or plastic width |
| | Polyethylene or plastic shape |
| | Polyethylene or plastic length |
| | Polyethylene or plastic height |
| | Polyethylene or plastic profile |
| | Polyethylene or plastic curvature |
| | Polyethylene or plastic angle |
| | Component stem width |
| | Component stem shape |
| | Component stem length |
| | Component stem height |
| | Component stem profile |
| | Component stem curvature |
| | Component stem position |
| | Component stem thickness |
| | Component stem angle |
| | Component peg width |
| | Component peg shape |
| | Component peg length |
| | Component peg height |
| | Component peg profile |
| | Component peg curvature |
| | Component peg position |
| | Component peg thickness |
| | Component peg angle |
| | Slope of an implant surface |
| | Number of sections, facets, or cuts on an implant surface |
| Glenoid Component(s) | One or more glenoid dimensions, e.g., superior-inferior |
| | diameter; anterior-posterior diameter; medio-lateral diameter, one |
| | or more oblique diameters |
| | glenoid reaming depth |
| | anatomic glenoid center point |
| | biomechanic glenoid center point such as center of rotation; |
| | glenoid angle (angle of inclination) |
| | glenoid cup position, e.g., anteversion, retroversion, |
| | rotation |
| | Composite glenoid dimensions (e.g., size, thickness or |
| | angle) |
| Humeral Component(s) | Humeral head, neck and diaphysis dimensions (head |
| | size/diameter) |
| | Humeral head or neck resection surface, region |
| | Humeral head or neck resection angle, region |
| | Humeral neck angle (cortical or endosteal) |
| | Humeral neck, stem geometry |
| | Humeral coating/texture |
| | Humeral anteversion or retroversion |
| | Humeral neck diameter (cortical or endosteal) |
| | Humeral shaft medio-lateral dimensions (cortical or |
| | endosteal) |
| | Humeral shaft anterior-posterior dimensions (cortical or |
| | endosteal) |
| | Humeral shaft length |
| | Humeral offset |
| | Humeral neck collar (and collar size/shape) |

Electronic systems and processes according to various embodiments of the disclosure can utilize computing capacity, including stand-alone and/or networked capacities, to determine and/or store data regarding the spatial aspects of surgically related items and virtual constructs or references, including body parts, implements, instrumentation, trial components, prosthetic components and anatomical, mechanical and/or rotational axes of body parts. Any or all of these may be physically or virtually connected to or incorporate any desired form of mark, structure, component, or other fiducial or reference device or technique which allows position and/or orientation of the item to which it is attached to be visually and/or tactily determined, as well as possibly sensed and tracked, either virtually or in physical space (e.g., for computation and/or display during a surgical operation), preferably in three dimensions of translations and varying degrees of rotation as well as in time, if desired. Systems and processes according to some embodiments can employ computing means to calculate and store references axes of body components such as in shoulder arthroplasty, for example the anatomical axis of the humerus and the retroversion reference axis.

If desired, various computing systems may employ patient-specific and/or patient-adapted data and computer models to track the position of instrumentation and osteotomy guides “real time” so that bone resections will locate the implant position optimally, which can include locations aligned with the anatomical axis. Furthermore, during trial reduction of the shoulder, such tracking systems can provide feedback on the balancing of the soft tissue in a range of motion and under stresses and can suggest or at least provide more accurate information than in the past about which ligaments the surgeon should release (or avoid releasing) in order to obtain correct balancing, alignment and stability. Systems and processes according to some embodiments can also suggest modifications to implant size, positioning, and other techniques to achieve optimal kinematics, either prior to surgery during the design and/or selection/modification process for implants, tools and/or procedural steps, or during the surgical procedure itself. Various systems can also include databases of information regarding tasks such as ligament balancing, in order to provide suggestions to the implant designer and/or surgeon based on performance of test results as automatically calculated by such systems and processes.

Reference points and/or data for obtaining measurements of a patient's joint, for example, relative-position measurements, length or distance measurements, curvature measurements, surface contour measurements, thickness measurements (in one location or across a surface), volume measurements (filled or empty volume), density measurements, and other measurements, can be obtained using any suitable technique. For example, one dimensional, two-dimensional, and/or three-dimensional measurements can be obtained using data collected from mechanical means, laser devices, electromagnetic or optical tracking systems, molds, materials applied to the articular surface that harden as a negative match of the surface contour, and/or one or more imaging techniques described above and/or known in the art. Data and measurements can be obtained non-invasively and/or preoperatively. Alternatively, measurements can be obtained intraoperatively, for example, using a probe or other surgical device during surgery.

In certain embodiments, reference points and/or measurements, such as those described above, can be processed using mathematical functions to derive virtual, corrected features, which may represent a restored, ideal or desired feature from which a patient-adapted implant component can be designed. For example, one or more features, such as surfaces or dimensions of a biological structure can be modeled, altered, added to, changed, deformed, eliminated, corrected and/or otherwise manipulated (collectively referred to herein as “variation” of an existing surface or structure within the joint). While it is described in the shoulder, these embodiments can be applied to any joint or joint surface in the body, e.g. a knee, hip, ankle, foot, toe, elbow, wrist, hand, and a spine or spinal joints.

Variation of the joint or portions of the joint can include, without limitation, variation of one or more external surfaces, internal surfaces, joint-facing surfaces, uncut surfaces, cut surfaces, altered surfaces, and/or partial surfaces as well as osteophytes, subchondral cysts, geodes or areas of eburnation, joint flattening, contour irregularity, and loss of normal shape. The surface or structure can be or reflect any surface or structure in the joint, including, without limitation, bone surfaces, ridges, plateaus, cartilage surfaces, ligament surfaces, or other surfaces or structures. The surface or structure derived can be an approximation of a healthy joint surface or structure or can be another variation. The surface or structure can be made to include pathological alterations of the joint. The surface or structure also can be made whereby the pathological joint changes are virtually removed in whole or in part.

Once one or more reference points, measurements, structures, surfaces, models, or combinations thereof have been selected or derived, the resultant shape can be varied, deformed or corrected. In certain embodiments, the variation can be used to select and/or design an implant component having an ideal or optimized feature or shape, e.g., corresponding to the deformed or corrected joint feature or shape. For example, in one application of this embodiment, the ideal or optimized implant shape reflects the shape of the patient's joint before he or she developed arthritis.

Alternatively or in addition, the variation can be used to select and/or design a patient-adapted surgical procedure to address the deformity or abnormality. For example, the variation can include surgical alterations to the joint, such as virtual resection cuts, virtual drill holes, virtual removal of osteophytes, and/or virtual building of structural support in the joint deemed necessary or beneficial to a desired final outcome for a patient.

In certain embodiments, imaging data collected from the patient, for example, imaging data from one or more of x-ray imaging, digital tomosynthesis, cone beam CT, non-spiral or spiral CT, non-isotropic or isotropic MRI, SPECT, PET, ultrasound, laser imaging, photo-acoustic imaging, is used to qualitatively and/or quantitatively measure one or more of a patient's biological features, one or more of normal cartilage, diseased cartilage, a cartilage defect, an area of denuded cartilage, subchondral bone, cortical bone, endosteal bone, bone marrow, a ligament, a ligament attachment or origin, menisci, labrum, a joint capsule, articular structures, and/or voids or spaces between or within any of these structures. The qualitatively and/or quantitatively measured biological features can include, but are not limited to, one or more of length, width, height, depth and/or thickness; curvature, for example, curvature in two dimensions (e.g., curvature in or projected onto a plane), curvature in three dimensions, and/or a radius or radii of curvature; shape, for example, two-dimensional shape or three-dimensional shape; area, for example, surface area and/or surface contour; perimeter shape; and/or volume of, for example, the patient's cartilage, bone (subchondral bone, cortical bone, endosteal bone, and/or other bone), ligament, and/or voids or spaces between them.

In certain embodiments, measurements of biological features can include any one or more of the illustrative measurements identified in Table 2.

TABLE 2
Exemplary patient-specific measurements of biological features
that can be used in the creation of a model and/or in the
selection and/or design of an implant component

| Anatomical feature | Exemplary measurement |
|---|---|
| Joint-line, | Location relative to proximal reference point |
| joint gap | Location relative to distal reference point |
| | Angle |
| | Gap distance between opposing surfaces in |
| | one or more locations |
| | Location, angle, and/or distance relative to |
| | contralateral joint |
| Soft tissue | Joint gap distance |
| tension and/or | Joint gap differential, e.g., medial to lateral |
| balance | |
| Medullary cavity | Shape in one or more dimensions |
| | Shape in one or more locations |
| | Diameter of cavity |
| | Volume of cavity |
| Subchondral bone | Shape in one or more dimensions |
| | Shape in one or more locations |
| | Thickness in one or more dimensions |
| | Thickness in one or more locations |
| | Angle, e.g., resection cut angle |
| Cortical bone | Shape in one or more dimensions |
| | Shape in one or more locations |
| | Thickness in one or more dimensions |
| | Thickness in one or more locations |
| | Angle, e.g., resection cut angle |
| | Portions or all of cortical bone perimeter at an |
| | intended resection level |
| Endosteal bone | Shape in one or more dimensions |
| | Shape in one or more locations |
| | Thickness in one or more dimensions |
| | Thickness in one or more locations |
| | Angle, e.g., resection cut angle |
| Cartilage | Shape in one or more dimensions |
| | Shape in one or more locations |
| | Thickness in one or more dimensions |
| | Thickness in one or more locations |
| | Angle, e.g., resection cut angle |
| Glenoid | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Anteversion or retroversion |
| | Portions or all of cortical bone perimeter at an |
| | intended resection level |
| | Resection surface at an intended resection level |
| Humeral head | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Anteversion or retroversion |
| | Portions or all of cortical bone perimeter at an |
| | intended resection level |
| | Resection surface at an intended resection level |
| Humeral neck | 2D and/or 3D shape of a portion or all |
| | Height in one or more locations |
| | Length in one or more locations |
| | Width in one or more locations |
| | Depth in one or more locations |
| | Thickness in one or more locations |
| | Angle in one or more locations |
| | Neck axis in one or more locations |
| | Curvature in one or more locations |
| | Slope in one or more locations and/or directions |
| | Angle, e.g., resection cut angle |
| | Anteversion or retroversion |
| | Arm length |
| | Portions or all of cortical bone perimeter at an |
| | intended resection level |
| | Resection surface at an intended resection level |
| Humeral shaft | 2D and/or 3D shape of a portion or all |
| | Height in one or more locations |
| | Length in one or more locations |
| | Width in one or more locations |
| | Depth in one or more locations |
| | Thickness in one or more locations |
| | Angle in one or more locations |
| | Shaft axis in one or more locations |
| | Curvature in one or more locations |
| | Angle, e.g., resection cut angle |
| | Anteversion or retroversion |
| | Arm length |
| | Portions or all of cortical bone perimeter at an |
| | intended resection level |
| | Resection surface at an intended resection level |

Depending on the clinical application, a single or any combination or all of the measurements described in Table 2 and/or known in the art can be used. Additional patient-specific measurements and information that be used in the evaluation can include, for example, joint kinematic measurements, bone density measurements, bone strength measurements, bone quality measurements, bone porosity measurements, identification of damaged or deformed tissues or structures, and patient information, such as patient age, weight, gender, ethnicity, activity level, and overall health status. Moreover, the patient-specific measurements may be compared, analyzed or otherwise modified based on one or more “normalized” or other patient model or models, or by reference to a desired database of anatomical features of interest. Any parameter mentioned in the specification and in the various Tables throughout the specification including anatomic, biomechanical and kinematic parameters can be utilized in the shoulder and other joints. Such analysis may include modification of one or more patient-specific features and/or design criteria for the implant to account for any underlying deformity reflected in the patient-specific measurements. If desired, the modified data may then be utilized to choose or design an appropriate implant to match the modified features, and a final verification operation may be accomplished to ensure the chosen implant is acceptable and appropriate to the original unmodified patient-specific measurements (i.e., the chosen implant will ultimately “fit” the original patient anatomy). In alternative embodiments, the various anatomical features may be differently “weighted” during the comparison process (utilizing various formulaic weightings and/or mathematical algorithms), based on their relative importance or other criteria chosen by the designer/programmer and/or physician.

In certain embodiments, bone cuts and implant shape including at least one of a bone-facing or a joint-facing surface of the implant can be designed or selected to achieve normal joint kinematics.

In certain embodiments, a computer program simulating biomotion of one or more joints, such as, for example, a shoulder joint, or a shoulder and elbow joint, can be utilized. In certain embodiments, patient-specific imaging data can be fed into this computer program. For example, a series of two-dimensional images of a patient's shoulder joint or a three-dimensional representation of a patient's shoulder joint can be entered into the program. Additionally, two-dimensional images or a three-dimensional representation of the patient's elbow joint (or other anatomical structures adjacent to the shoulder, such as the torso or neck) may be added.

Alternatively, patient-specific kinematic data, for example obtained in a motion or gait lab, can be fed into the computer program. Alternatively, patient-specific navigation data, for example generated using a surgical navigation system, image guided or non-image guided can be fed into the computer program. This kinematic or navigation data can, for example, be generated by applying optical or RF markers to the relevant limb(s) and by registering the markers and then measuring limb movements, for example, flexion, extension, abduction, adduction, rotation, and other limb movements.

Optionally, other data including anthropometric data may be added for each patient. These data can include but are not limited to the patient's age, gender, weight, height, size, body mass index, and race. Desired limb alignment and/or deformity correction can be added into the model. The position of bone cuts on one or more articular or other surfaces as well as the intended location of implant bearing surfaces on one or more articular surfaces can be entered into the model.

A patient-specific biomotion model can be derived that includes combinations of parameters listed above. The biomotion model can simulate various activities of daily life including normal gait, stair climbing, descending stairs, running, kneeling, squatting, sitting and any other physical activity, as well as shoulder and/or arm-specific motions such as shoulder flexion, extension, scaption, abduction, horizontal abduction, horizontal adduction, external rotation, internal rotation, and various other lifting, rotating and/or pushing/pulling action such as arm raises, push-ups, pull-ups and the like. The biomotion model can start out with standardized activities, typically derived from reference databases. These reference databases can be, for example, generated using biomotion measurements using force plates and motion trackers using radiofrequency or optical markers and video equipment.

The biomotion model can then be individualized with use of patient-specific information including at least one of, but not limited to the patient's age, gender, weight, height, body mass index, and race, the desired limb alignment or deformity correction, and the patient's imaging data, for example, a series of two-dimensional images or a three-dimensional representation of the joint for which surgery is contemplated.

An implant shape including associated bone cuts generated in the preceding optimizations, for example, limb alignment, deformity correction, bone preservation on one or more articular surfaces, can be introduced into the model. Table 3 includes an exemplary list of parameters that can be measured in a patient-specific biomotion model.

TABLE 3
Parameters measured in a patient-specific biomotion model for various implants

| Joint implant | Measured Parameter |
|---|---|
| Shoulder or other | Internal and external rotation of one or more articular surfaces |
| joint | |
| Shoulder or other | Flexion and extension angles of one or more articular surfaces |
| joint | |
| Shoulder or other | Anterior slide and posterior slide of at least one or more articular surfaces |
| joint | during flexion or extension, abduction or adduction, elevation, internal or |
| | external rotation |
| Shoulder or other | Joint laxity throughout the range of motion |
| joint | |
| Shoulder or other | Contact pressure or forces on at least one or more articular surfaces, e.g. an |
| joint | acetabulum and a femoral head, a glenoid and a humeral head |
| Shoulder or other | Forces between the bone-facing surface of the implant, an optional cement |
| joint | interface and the adjacent bone or bone marrow, measured at least one or |
| | multiple bone cut or bone-facing surface of the implant on at least one or |
| | multiple articular surfaces or implant components. |
| Shoulder or other | Ligament location, e.g. transverse ligament, glenohumeral ligaments, |
| joint | retinacula, joint capsule, estimated or derived, for example using art imaging |
| | test. |
| Shoulder or other | Ligament tension, strain, shear force, estimated failure forces, loads for |
| joint | example for different angles of flexion, extension, rotation, abduction, |
| | adduction, with the different positions or movements optionally simulated in |
| | a virtual environment. |
| Shoulder or other | Potential implant impingement on other articular structures, e.g. in high |
| joint | flexion, high extension, internal or external rotation, abduction or adduction |
| | or elevation or any combinations thereof or other angles/positions/ |
| | movements. |

The above list is not meant to be exhaustive, but only exemplary. Any other biomechanical parameter known in the art can be included in the analysis.

The resultant biomotion data can be used to further optimize the implant design with the objective to establish normal or near normal kinematics. The implant optimizations can include one or multiple implant components. Implant optimizations based on patient-specific data including image based biomotion data include, but are not limited to:
Changes to external, joint-facing implant shape in coronal plane
Changes to external, joint-facing implant shape in sagittal plane
Changes to external, joint-facing implant shape in axial plane
Changes to external, joint-facing implant shape in multiple planes or three dimensions
Changes to internal, bone-facing implant shape in coronal plane
Changes to internal, bone-facing implant shape in sagittal plane
Changes to internal, bone-facing implant shape in axial plane
Changes to internal, bone-facing implant shape in multiple planes or three dimensions
Changes to one or more bone cuts, for example with regard to depth of cut, orientation of cut

Any single one or combinations of the above or all of the above on at least one articular surface or implant component or multiple articular surfaces or implant components.

When changes are made on multiple articular surfaces or implant components, these can be made in reference to or linked to each other. For example, in the shoulder, a change made to a humeral bone cut based on patient-specific biomotion data can be referenced to or linked with a concomitant change to a bone cut on an opposing glenoid/scapular surface or structure. For example, if less humeral bone is resected, the computer program may elect to resect more glenoid bone.

Similarly, if a humeral implant shape is changed, for example on an external surface, this may be accompanied by a change in the glenoid component shape. This is, for example, particularly applicable when at least portions of the glenoid bearing surface negatively-match the humeral head joint-facing surface.

Similarly, if the footprint of a glenoid implant is broadened, this can be accompanied by a widening of the bearing surface of a humeral component. Similarly, if a humeral implant shape is changed, for example on an external surface, this can be accompanied by a change in the glenoid component shape.

Such linked changes can be particularly relevant to shoulder implants. In a shoulder, if a glenoid implant shape is changed, for example on an external surface, this can be accompanied by a change in a humeral component shape. This is, for example, particularly applicable when at least portions of the humeral bearing surface negatively-match the glenoid joint-facing surface, or vice-versa.

Any combination is possible as it pertains to the shape, orientation, and size of implant components on two or more opposing surfaces.

By optimizing implant shape in this manner, it is possible to establish normal or near normal kinematics. Moreover, it is possible to avoid implant related complications, including but not limited to tissue or component impingement in high flexion or rotation, and other complications associated with existing implant designs. Since traditional implants follow a one-size-fits-all approach, they are generally limited to altering only one or two aspects of an implant design. However, with the design approaches described herein, various features of an implant component can be designed for an individual to address multiple issues, including issues associated with various particularized motion. For example, designs as described herein can alter an implant component's bone-facing surface (for example, number, angle, and orientation of bone cuts), joint-facing surface (for example, surface contour and curvatures) and other features (for example, implant height, width, and other features) to address patient-specific issues.

Biomotion models for a particular patient can be supplemented with patient-specific finite element modeling or other biomechanical models known in the art. Resultant forces in the shoulder joint can be calculated for each component for each specific patient. The implant can be engineered to the patient's load and force demands. For instance, a 125 lb. patient may not need a glenoid insert as thick as a patient weighing 280 lbs. Similarly, the polyethylene can be adjusted in shape, thickness and material properties for each patient. For example, a 3 mm polyethylene insert can be used in a light patient with low force and a heavier, stronger or more active patient may require a different implant size and/or design, such as an 8 mm thick polymer insert or similar device.

The present disclosure describes improved patient-specific or patient engineered shoulder implant components, including glenoid implants, templates, alignment guides and apparatus (hereinafter “glenoid templates”) and associated methods that desirably overcome and/or address various disadvantages of existing systems. The present disclosure may also facilitate the partial replacement of shoulder joints (e.g., the retention of a natural humeral head with a glenoid replacement or resurfacing component, or retention of a natural glenoid surface with a humeral resurfacing or replacement component) as well as resurfacing and/or repairing of a natural glenoid surface. In addition, the disclosure can be used in association with anchoring and/or positioning of implant components into and/or adjacent to other bones having limited, damaged, degraded and/or unusual support structures.

The embodiments described herein include advancements in or that arise out of the area of patient-adapted articular implants that are tailored to address the needs of individual, single patients. Such patient-adapted articular implants offer advantages over the traditional one-size-fits-all approach, or a few-sizes-fit-all approach. The advantages include, for example, better fit, more natural movement of the joint, reduction in the amount of bone removed during surgery and a less invasive procedure. Such patient-adapted articular implants can be created from images of the patient's joint. Based on the images, patient-adapted implant components can be selected and/or designed to include features (e.g., surface contours, curvatures, widths, lengths, thicknesses, and other features) that match existing features in the single, individual patient's joint as well as features that approximate an ideal and/or healthy feature that may not exist in the patient prior to a procedure. Moreover, by altering the design approach to address several implant design issues, several non-traditional design and/or implantation approaches have been identified that offer improvements over traditional implant designs.

Patient-adapted features can include patient-specific and/or patient-engineered. Patient-specific (or patient-matched) implant component or guide tool features can include features adapted to match one or more of the patient's biological features, for example, one or more biological/anatomical structures, alignments, kinematics, and/or soft tissue features. Patient-engineered (or patient-derived) features of an implant component can be designed and/or manufactured (e.g., preoperatively designed and manufactured) based on patient-specific data to substantially enhance or improve one or more of the patient's anatomical and/or biological features.

The patient-adapted (e.g., patient-specific and/or patient-engineered) implant components and guide tools described herein can be selected (e.g., from a library), designed (e.g., preoperatively designed including, optionally, manufacturing the components or tools), and/or selected and designed (e.g., by selecting a blank component or tool having certain blank features and then altering the blank features to be patient-adapted). Moreover, related methods, such as designs and strategies for resectioning a patient's biological structure also can be selected and/or designed. For example, an implant component bone-facing surface and a resectioning strategy for the corresponding bone-facing surface can be selected and/or designed together so that an implant component's bone-facing surface match or otherwise conform to or accommodate the resected surface(s). In addition, one or more guide tools optionally can be selected and/or designed to facilitate the resection cuts that are predetermined in accordance with resectioning strategy and implant component selection and/or design.

In certain embodiments, patient-adapted features of an implant component, guide tool or related method can be achieved by analyzing imaging test data and selecting and/or designing (e.g., preoperatively selecting from a library and/or designing) an implant component, a guide tool, and/or a procedure having a feature that is matched and/or optimized for the particular patient's biology. The imaging test data can include data from the patient's joint, for example, data generated from an image of the joint such as x-ray imaging, cone beam CT, digital tomosynthesis, and ultrasound, a MRI or CT scan or a PET or SPECT scan, which can be processed to generate a varied or corrected version of the joint or of portions of the joint or of surfaces within the joint. Certain embodiments provide methods and/or devices to create a desired model of a joint or of portions or surfaces of a joint based, at least partially, on data derived from the existing joint. For example, the data can also be used to create a model that can be used to analyze the patient's joint and to devise and evaluate a course of corrective action. The data and/or model also can be used to design an implant component having one or more patient-specific features, such as a surface or curvature.

In one aspect, embodiments described herein provide a primary articular implant component that includes (a) an inner, joint-facing surface and an outer, bone-facing surface. The inner, joint-facing surface can include a bearing surface. The outer, bone facing surface can include one or more patient-engineered bone cuts and/or other features selected and/or designed from patient-specific data. In certain embodiments, the patient-engineered bone cuts can be selected and/or designed from patient-specific data to minimize the amount of bone resected in one or more corresponding predetermined resection cuts and/or maximize the stability of the implant component. In certain embodiments, the patient-engineered bone cuts substantially negatively-match one or more predetermined resection cuts. The predetermined resection cuts can be made at a first depth that allows, in a subsequent procedure, removal of additional bone to a second depth required for a traditional implant component (which may be employed as a revision component, if desired).

In certain embodiments, the primary articular implant component can include an implant component thickness in one or more regions that is selected and/or designed from patient-specific data to minimize the amount of bone resected. The one or more regions can comprise the implant component thickness perpendicular to a planar bone cut and between the planar bone cut and the joint-surface of the implant component.

In other aspects, embodiments described herein provide methods for minimizing resected bone from, and/or methods for making an articular implant for, a single patient in need of an articular implant replacement procedure. These methods can include (a) identifying unwanted tissue from one or more images of the patient's joint; (b) identifying a combination of resection cuts and implant component features that remove the unwanted tissue and also provide maximum bone preservation; and (c) selecting and/or designing for the patient a combination of resection cuts and implant component features that provide removal of the unwanted tissue and maximum bone preservation. In certain embodiments, the unwanted tissue is diseased tissue or deformed tissue.

In certain embodiments, various procedural steps can include designing for an individual patient a combination of resection cuts and implant component features that provide removal of unwanted tissue and maximum bone preservation. Designing can include manufacturing. Moreover, the implant component features can include one or more of the features selected from the group consisting of implant thickness, bone cut number, bone cut angles, and/or bone cut orientations.

In certain embodiments, a measure of bone preservation can include a total volume of bone resected, a volume of bone resected from one or more resection cuts, a volume of bone resected to fit one or more implant component bone cuts, an average thickness of bone resected, an average thickness of bone resected from one or more resection cuts, an average thickness of bone resected to fit one or more implant component bone cuts, a maximum thickness of bone resected, a maximum thickness of bone resected from one or more resection cuts and/or a maximum thickness of bone resected to fit one or more implant component bone cuts.

In certain embodiments, a minimum implant component thickness or other dimension/feature also can be established. For example, various procedural steps can include identifying a minimum implant component thickness for an individual patient. An additional step can include identifying a combination of resection cuts and/or implant component features that provide a minimum implant thickness determined for an individual patient. Another step can include selecting and/or designing the combination of resection cuts and/or implant component features that provides at least a minimum implant thickness for the individual patient. The minimum implant component thickness can be based on one or more of the humeral and/or glenoid/scapular size or patient weight or strength.

In various embodiments, implant components can include one or more outer, bone-facing surface(s) designed to negatively-match one or more bone surfaces that were cut, for example based on pre-determined geometries or based on patient-specific geometries. In certain embodiments, an inner joint-facing surface can include at least a portion that substantially negatively-matches a feature of the patient's anatomy and/or an opposing joint-facing surface of a second implant component. In certain embodiments, by creating negatively-matching component surfaces at a joint interface, the opposing surfaces may not have an anatomic or near-anatomic shape, but instead may be negatively-matching or near-negatively-matching to each other. This can have various advantages, such as reducing implant and joint wear and providing more predictable and/or controllable joint movement.

In various embodiments, implant components may be designed and/or selected to include one or more patient-specific curvatures or radii of curvature in one dimension or direction, and one or more standard or engineered curvatures or radii of curvature in a second dimension or direction. Such features may be included on a single individual joint component, or various combinations of such features can be complementary and/or mirrored on opposing implant components.

The present disclosure includes patient-specific alignment guides and associated orthopedic devices adapted for use in a shoulder joint. The alignment guide can include a cap or other structure having a three-dimensional engagement surface customized using patient-specific image data in a pre-operative plan by computer imaging to be complementary and closely mate and/or conform to a humeral head of a proximal humerus of a patient. The alignment guide can include one or more tubular or other elements extending from the cap, which desirably define one or more longitudinal guiding bore(s) for guiding alignment pins or other instruments at patient-specific positions and/or orientations determined in the pre-operative plan. The orientation feature(s) can be designed to orient the cap relative to the humeral head when the orientation feature(s) are aligned with various landmarks of the proximal humerus and/or glenoid/scapula. In at least one alternative embodiment, an alignment guide can include a surface feature, such as a void, osteophyte, surface variation and/or other unique anatomical “irregularity” to assist with alignment and/or desired positioning of the guide, such as a tab extending from the cap which is adapted or configured to be at least partially received into a bicipital groove of the proximal humerus.

In at least one exemplary embodiment, a patient-specific glenoid implant assembly can include a patient-specific and/or patient-engineered scapular anchor that is selected, constructed and/or modified using patient anatomical data, the anchor being connected or otherwise attached to a standard, modular, patient-specific and/or patient-engineered glenoid articulating component. In various embodiments, the scapular anchor may be designed and/or selected/modified using patient anatomical data modeled using a computer or other electronic processing equipment. The glenoid prosthesis can include a tray or bearing “shell” (e.g., somewhat similar to an acetabular shell of a hip replacement prosthesis) for accommodating the head or prosthetic ball of the humerus on an inner face and a patient-specific and/or patient-adapted anchor, stem or projection extending at an angle from an outer face of the tray to engage the anchor within a defined and/or created canal in the lateral border of the scapula, which can facilitate anchoring of the glenoid prosthesis to and within the scapula.

The present embodiments of the present disclosure may be patient-specific or patient engineered for each surgical patient, with one or more of each glenoid implant and associated glenoid template including features that are tailored to an individual patient's joint morphology. In at least one embodiment of the present disclosure, the system may be designed as an assembly that comprises a patient specific scapular anchor, a patient-specific glenoid implant and one or more patient-specific glenoid templates. In various alternative embodiments, instruments designed and/or selected/modified according to various teachings of the present disclosure may include surfaces and/or features that facilitate implantation of shoulder implant components. The instrument surfaces can include patient-specific features which conform to the actual diseased joint surfaces presented by the patient. The physician may use these instruments to align and direct surgical cuts, to prepare the patient to receive an otherwise standard and/or conventional joint component (some or all of which may include features that are patient-specific, patient-adapted and/or standard, or combinations thereof) of either “standard” or “reverse” shoulder implant configurations.

In various embodiments, portions of the glenoid template assembly can be uniquely tailored to an individual patient's anatomy, which may require images taken from the subject. The manufacturer can then design the patient-specific glenoid template assembly using the joint image from a patient or subject, wherein the image may include both normal cartilage or bone or diseased cartilage or bone; reconstructing dimensions of the diseased cartilage or bone surface to correspond to normal cartilage or bone (using, for example, a computer system); and designing the glenoid template to exactly or substantially match the perimeter dimensions of the resected glenoid surface, the normal cartilage surface, a healthy cartilage surface, a subchondral bone surface, and/or various combinations thereof (including height, width, length, curvature, rotation, medial/lateral, and posterior/anterior angles). The image can be, for example, an intraoperative image including a surface and/or feature detection method using any techniques known in the art, e.g., mechanical, optical, ultrasound, and known devices such as MRI, CT, ultrasound, and other image techniques known in the art. The images can be 2D or 3D or combination thereof to specifically design the glenoid template assembly.

In various embodiments, a plurality of glenoid templates may be utilized in an individual surgical procedure, with each glenoid template using various anatomical features of the glenoid and/or surrounding bone surface(s), either natural and/or resected (including those resected surfaces created using, for example, previous glenoid templates as guides), as alignment guides and/or other features accommodated by various corresponding surfaces of the template.

In various alternative embodiments, various individual components of the implant, the anchor and/or the template may comprise patient-specific, patient-engineered and/or standard sized features, such as varying posterior/anterior angles and/or orientations, varying cephalad/caudal angles and/or orientations, various cup and/or inner/outer surface radii and/or curvatures, and/or other varying dimensions. Each template can be designed to match one or more corresponding features of a patient-specific shoulder implant prosthesis and/or shoulder trial prosthesis (if any). The manufacturer may make different sizes available should the surgeon need to make adjustments to the resected humerus and/or glenoid/scapula.

In various embodiments, the template may include one or more integrated or modular drill and/or reamer guides. In at least one exemplary embodiment, the drill guide may be modular and have a quick connect/disconnect mechanism to the template when the surgeon is prepared to drill and/or ream the scapular canal and insert the scapular anchor. The drill guide may be sized to accommodate a “one-size fits all” drill, reamer or other tools, or the drill guide may be designed to accommodate and/or guide/limit one or more of several standard sizes for the surgeon to use. The drill guide may be integrated into the template to provide more of a positive stop for the surgeon when using the drill.

In other alternative embodiments, a portion of the template may form some portion of the glenoid and/or humeral implant component, with at least a portion of the template including an integrated or modular docking arrangement to accommodate various surgical tool guides for preparing some or all of the glenoid surface, the glenoid cavity and/or the scapular anchoring canal. Once the desired surgical preparation has been completed, the tool guide(s) may be removed by the surgeon and the remainder of the glenoid component (and/or scapular anchor or humeral components) can be secured to the template portion as desired.

In various embodiments, the glenoid component can include a metallic portion and a non-metallic portion, such as a metal backing plate or “tray” and a polyethylene insert attaching thereto. The backing plate may be secured directly to the prepared glenoid surface, and the poly insert attached to the joint-facing inner portion of the plate, in a manner similar to a tibial tray and polyethylene insert(s) of a knee arthroplasty implant. In various embodiments, multiple poly inserts of varying thicknesses, shapes, curvatures and/or sizes, including differing rim geometries, orientations and/or surface configurations, can be included and accommodated by a single metallic glenoid tray, thereby allowing the physician to modify the ultimate performance of the TSA implant (or portions thereof) during the surgical procedure.

Many surgical procedures require a wide array of instrumentation and other surgical items. Such items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes, wrenches, trial implants and cutting guides. In many surgical procedures, including orthopedic procedures, it may be desirable to employ patient-specific and/or patient-adapted image data and computerized modeling to optimize the design and/or selection/modification of one or more features of various instruments and implants to facilitate their use in surgical procedures. In some embodiments, an exemplary surgical instrument can be a reamer, a resection guide, a cutting block or a probe having one or more features designed and/or selected using patient-specific and/or patient-adapted image information and computerized models. In some more particular embodiments, the surgical instrument can comprise a humeral reamer or a glenoid reamer.

In at least one alternative embodiment, the various surgical tools and implant components described herein can include a computer-aided surgical navigation system with sensing capabilities (such as, for example, fiducial markers attached to instruments and/or anatomical locations) in a surgery on a shoulder, including a total shoulder arthroplasty. Systems and processes according to some embodiments of the disclosure could track various body parts such as a humerus and/or a glenoid/scapula, to which navigational sensors may be implanted, attached or associated physically, virtually or otherwise. Such systems and processes could employ position and/or orientation tracking sensors such as infrared sensors acting stereoscopically or other sensors acting in conjunction with navigational references to track positions of body parts, surgery-related items such as implements, instrumentation, trial prosthetics, prosthetic components, and virtual constructs or references such as rotational axes which have been calculated and stored based on designation of bone landmarks. Sensors, such as cameras, detectors, and other similar devices, could be mounted overhead with respect to body parts and surgery-related items to receive, sense, or otherwise detect positions and/or orientations of the body parts and surgery-related items. Processing capability such as any desired form of computer functionality, whether standalone, networked, or otherwise, could take into account the position and orientation information as to various items in the position sensing field (which may correspond generally or specifically to all or portions or more than all of the surgical field) based on sensed position and orientation of their associated navigational references, or based on stored position and/or orientation information. The processing functionality could correlate this position and orientation information for each object with stored information, such as a computerized fluoroscopic imaged file, a wire frame data file for rendering a representation of an instrument component, trial prosthesis or actual prosthesis, or a computer generated file relating to a reference, mechanical, rotational or other axis or other virtual construct or reference. Such information could be used to design and/or select/modify implant components and/or tools, as well as display position and orientation of these objects on a rendering functionality, such as a screen, monitor, or otherwise, in combination with image information or navigational information such as a reference, mechanical, rotational or other axis or other virtual

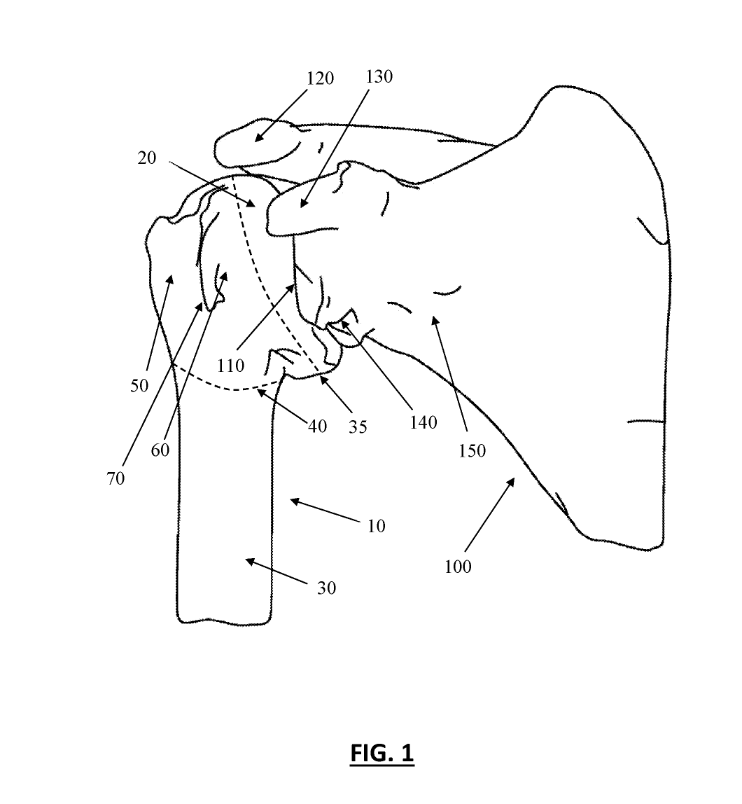

FIG. 1 depicts a humerus 10 and a scapula 100 of an exemplary shoulder joint illustrated schematically to indicate various features and landmarks. The humerus 10 includes a humeral head 20, a shaft 30, an anatomical neck 35, a surgical neck 40, a greater tuberosity or tubercle 50, a lesser tuberosity 60 and a bicipital groove 70 between the greater and lesser tuberosities. The scapula 100 includes a glenoid cavity 110 (opposing the humeral head 20), an acromion 120, a coracoid process 130, an infraglenoid tubercle 140 and a subscapular fossa 150.

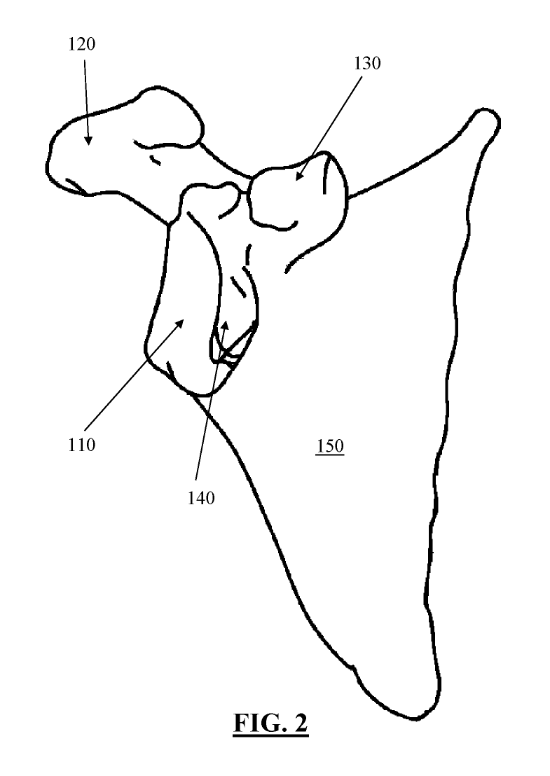

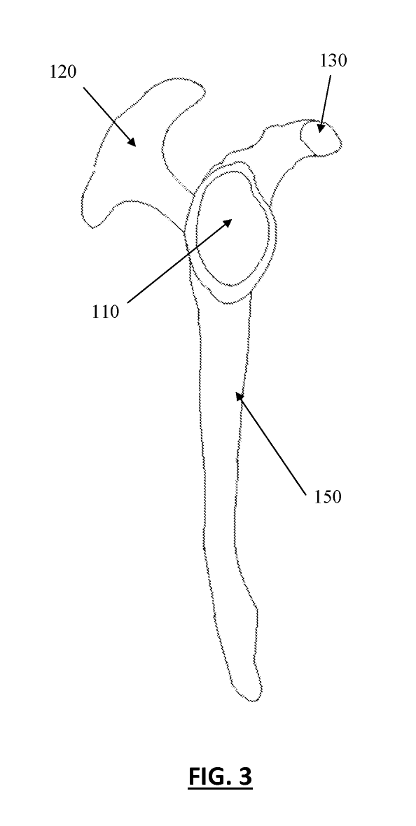

FIGS. 2 and 3 depict partial front and side views, respectively, of the scapula of FIG. 1. In these views, the glenoid cavity 110 can be clearly seen, as well as the acromion 120, the coracoid process 130, the infraglenoid tubercle 140 and the subscapular fossa 150. As can best be seen in FIGS. 2 and 3, the subscapular fossa 150 is typically a relatively broad, thin plate of bone, and this relative “thinness” in the anterior/posterior direction can significantly limit the scapula's ability to properly support a standard stem or other anchoring implant component as compared to other types of joint implant components (e.g., a tibial or femoral stem such as those used to support knee implant components).

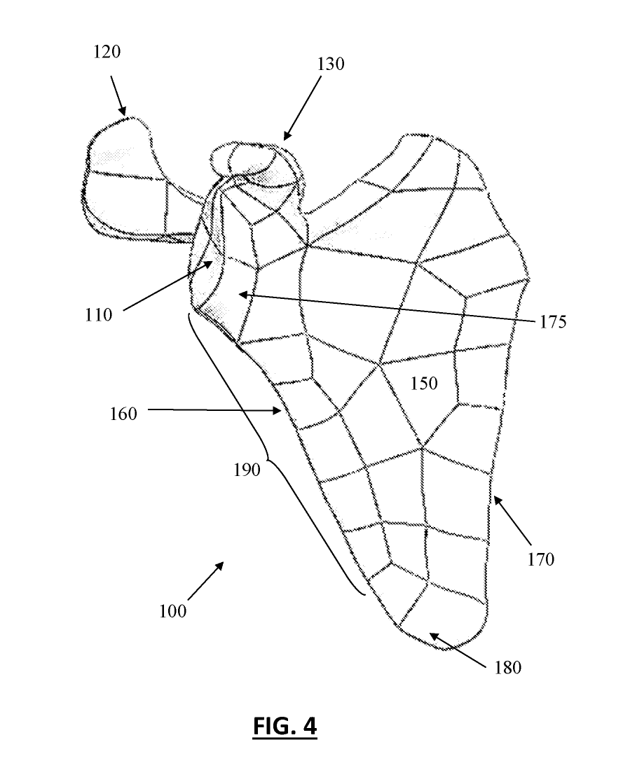

FIG. 4 depicts an exemplary 3-dimensional wire frame drawing of a scapula 100, showing various portions of the scapula, including the lateral border 160 and the medial border 170 of the subscapular fossa 150. Depending upon the patient's natural anatomy, a portion of the scapula adjacent the lateral border 160 may be naturally thicker (along an anterior to posterior measurement direction) relative to the remainder of the scapular fossa 150, with the thickened section 190 typically extending from the scapular neck 175 towards the interior angle 180.

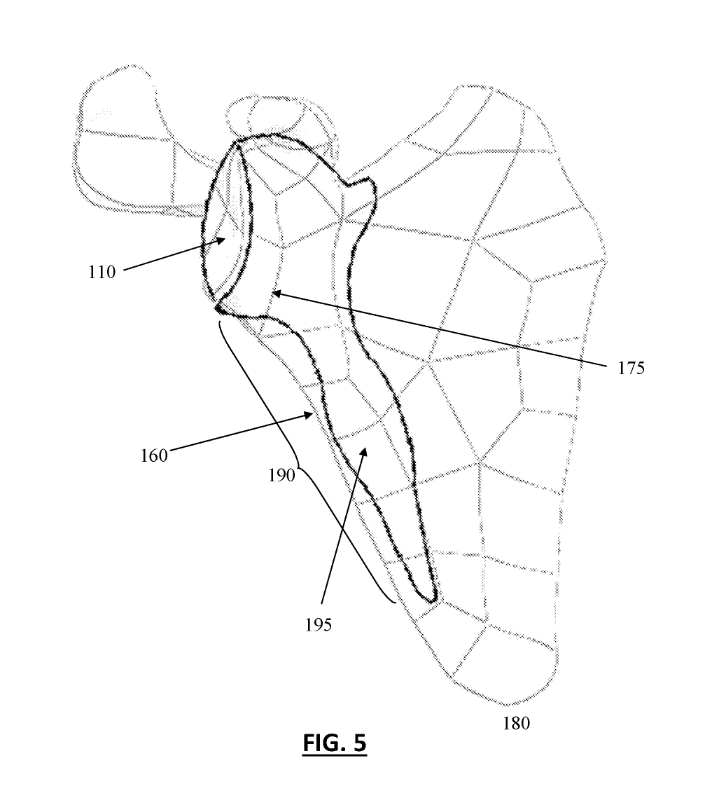

In various embodiments, the thickened section 190 of the lateral scapula fossa can be imaged and modeled using patient-specific data, to identify one or more cavities or canals 195 and/or other anatomical features (or sufficient bony structures that can be safely modified to create such cavities or canals) that can be utilized to facilitate anchoring or other fixation of one or more implant components, such as, for example, a glenoid implant. FIG. 5 depicts one exemplary glenoid canal 195 that has been modeled using anatomical image of the scapula of FIG. 4. In this embodiment, the canal 195 extends from the glenoid 110, through the scapular neck 175 and along the lateral margin 160 towards the interior angle 180.

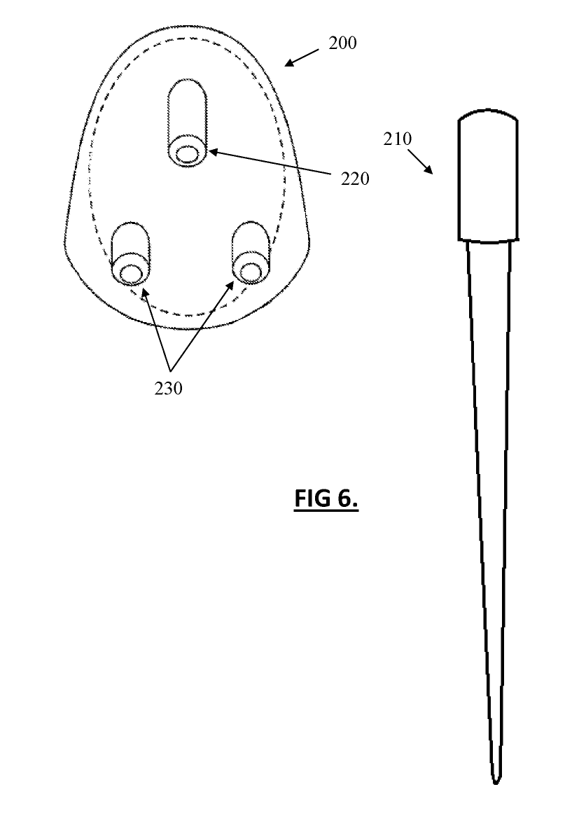

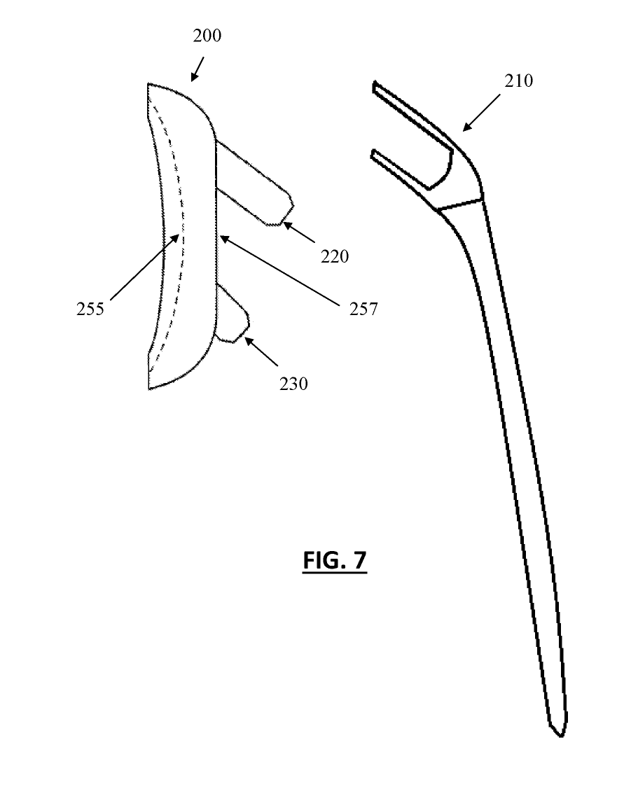

FIGS. 6 and 7 depict medial and side views, respectively, of one embodiment of an exemplary glenoid implant component 200, with an associated scapular anchor or stem 210 which can be configured to connect to one or more engagement structures 220 extending from a medial side 257 of the implant 200. In various embodiments, the size, shape and/or other configuration(s) of the glenoid component 200, including the configuration, shape and/or positioning of the engagement structure as well as any additional anchoring protrusions 230, can be determined based upon patient-specific anatomical information obtained prior to the surgery, which can be utilized to determine an appropriate size, shape and/or other configuration of the glenoid component to fit within the glenoid socket of the treated shoulder joint. In various embodiments, patient-specific data can be used in conjunction with modeling of the shoulder anatomy (as well as the use of non-patient sources such as databases of similar patients and/or individuals from a given patient population and/or normalized data including general engineering and/or kinematic modeling data) to derive an improved, desired and/or optimal configuration(s) for one or more features of the joint replacement implant, which can then be incorporated into (and/or selected into) the glenoid and/or humeral components as desired. In the exemplary embodiment, the glenoid component has been chosen to have a desired shape and size to fit within a prepared glenoid cavity.

In one exemplary embodiment, two-dimensional image data can be programmed into a software program such as MIMICS™ (commercially available from Materialise HQ of Leuven, Belgium) which can take MRI or CT data and create a 3-dimensional image of the glenoid and scapular spine that can be manipulated on the computer screen. The computer (or a user, if manual input is desired) can define three or more points, including a glenoid center point in the center of the glenoid articular surface, a junction point along the ridge of the scapular spine where the medial border and scapular spine meet, and an inferior point at the most distal end of the scapular spine. These reference points can be used to define a coronal plane, and then a transverse plane orthogonal to the coronal plane can be created through the glenoid center point and scapular spine junction point. Next, a sagittal plane can be defined in an orientation orthogonal to the previous coronal and transverse planes, and can be centered on the center point of the glenoid. This approach facilitates the definition of a reference anatomic axis at the intersection of the transverse and sagittal planes. Such steps can be performed using a conventional software package such as Creo Elements/Pro.

In one exemplary embodiment, in order to reproduce a normal anatomic orientation of a glenoid articulating surface after total shoulder arthroplasty, an ideal orientation of the glenoid component can be approximately 4 degrees of superior inclination and approximately 1 degree of retroversion. Desirably, the glenoid component and associated scapular anchor/fixation pegs/stems will be designed to achieve such an orientation while accommodating the natural anatomy and available bone stock. For example, the glenoid component may be re-centered or medialized; the anchoring mechanisms (e.g., fixation pegs or stems) may be sized, shaped and/or located to accommodate the patient's available bone stock and other natural anatomy.

If desired, a glenoid guide tool can designed and/or selected to include a set of apertures that can function as windows to observe tissue and or as guide to direct cutting tools into the glenoid. For example, the guide tool may carry a center hole for a drill bit to pass there-through for creating a cavity to accommodate a central peg of a glenoid component, or a slot to facilitate cutting a keel slot through the guide tool to accommodate a keel of a glenoid component. The orientation of the center aperture may be normal to a glenoid component plane and can be positioned and/or centered based on a pre-operative plan. Peripheral holes in the guide tool can be added to match any peripheral pegs/keels/screws or the like that the glenoid component may require, which may include a plurality of such holes that allow the surgeon to use one or more of such holes as desired and needed. The peripheral holes can be employed to create various voids to accommodate pegs, etc., which can result in various orientations of the glenoid component (such as rotation about the central axis for the glenoid component). The location of the holes or windows or slots can be used to determine the rotation of the glenoid component, as desired. In various embodiments, the location of viewing slot(s) or other openings may be defined for the instrument based on instrument design and/or anatomical features. Such slots or other openings (as well as other visual or tactile indicia) can be positioned so that they can be observed and/or felt by the physician during the surgery relative to one or more anatomical surfaces so that the presence or absence of a bony surface or other feature in and/or adjacent to the window helps verify the seating and/or orientation of the tool. Various embodiments may include an extending handle or other feature that is directed away (i.e., superiorly or anteriorly) from the axis of the peg/keel, which can facilitate other surgical tools, such as a drill, to access the guide tool.

FIGS. 6 and 7 depict one exemplary embodiment of a scapular anchor 210 that can be designed, selected and/or modified to secure and/or supplement fixation of a glenoid component to the scapula. In various embodiments, the size, shape and/or other configuration(s) of the scapular anchor 210 can be determined based upon patient-specific anatomical information obtained prior to the surgery, which can be utilized to determine an appropriate size, shape and/or other configuration of a scapular anchor to fit within a cavity, canal or other anatomic feature of the scapula of the treated shoulder joint. In various alternative embodiments, patient-specific data can be used in conjunction with modeling of the shoulder anatomy (as well as the use of non-patient sources such as databases of similar patients and/or individuals from a given patient population and/or normalized data including general engineering and/or kinematic modeling data) to derive an improved, desired and/or optimal configuration(s) for one or more features of the joint replacement implant, which can then be incorporated into (and/or selected into) the scapular anchor, if desired and/or necessary. Because the scapular anatomy (as well as relevant canal anatomy) can widely vary among the general population, the use of patient data and patient modeling data can be particularly useful in determining a proper alignment, size and shape of the scapular anchor to provide sufficient anchoring of the glenoid component without fracturing, penetrating and/or otherwise unnecessarily weakening the scapular bone. In the exemplary embodiment, the scapular anchor has been designed to have an engagement portion, a neck distance, a neck angle, a shaft diameter, a shaft length and a shaft curvature that has been particularized to the patient's specific thickened section 190 adjacent the lateral margin of the scapula (as depicted in FIG. 5).

In various embodiments, exemplary dimensions for one or more diameters of a scapular anchor can range from about 2 mm to about 10 mm. In other embodiments, the length of the scapular anchor can vary, with at least one embodiment including an anchor of less than about 200 mm.

FIG. 8 depicts a side view of the glenoid component 200 and scapular anchor 210 of FIGS. 6 and 7, with the anchor 210 docked with and secured to the glenoid component 200 using a threaded screw 240 or other connection mechanism known in the art. Also shown is an insert 250, which can fit within a recess 255 in a lateral face (joint-facing, in this embodiment) of the glenoid component and desirably form an articulating surface that interacts with the natural humeral head and/or a humeral joint replacement surface. In various embodiments, the insert can comprise a polymer, metal or ceramic material. As previously noted, in various embodiments the glenoid component can comprise a metallic backing plate or “tray” and a polyethylene insert attaching thereto. The backing plate may be secured directly to the prepared glenoid surface, and the poly insert attached to the joint-facing portion of the plate, in a manner similar to a tibial tray and polyethylene insert of a knee implant. In various embodiments, multiple poly inserts of varying thicknesses, shapes and/or sizes, including differing rim geometries and/or surface configurations, can be included and/or accommodated by a single metallic glenoid tray, thereby allowing the physician to modify the ultimate performance of the TSA implant (or portions thereof) during the surgical procedure. In various embodiments, the insert may form a primary articulating surface, with a peripheral rim of the glenoid component 200 forming a secondary articulating surface, in a manner similar to a glenoid surface and labrum of the natural shoulder joint.

The tray and anchoring stem can be modular, or can be constructed and/or implanted as a one-piece implant. In a modular prosthesis system, a combination of a tray and anchor can be chosen from a variety of shapes and sizes, including one or more components having patient-specific and/or patient-adapted features. In various embodiments, one or more components (e.g., the glenoid tray or “shell”) can be selected from previously manufactured and/or stockpiled components so as to most closely match (or approximate in some desired manner) the natural anatomy of the joint undergoing arthroplasty, while other components (e.g., the scapular anchor and/or connection mechanisms) can be designed and/or selected/modified using patient-specific data and/or patient-adapted modeling. In various embodiments, a wearing surface or other feature(s) of an insert can be secured to the inner concave surface of the tray.

In various embodiments, the glenoid prosthesis may comprise a tray component having an outer convex face configured to contact a resected surface of the scapula, and an anchor or stem configured to extend from the component into a passage or canal in a lateral border of the scapula, with the anchor configured to engage within the canal and thereby anchor the glenoid prosthesis to the scapula. If desired, the tray component can incorporate an opening there-through such that the anchor can be inserted into the canal through the opening, either before or after implantation of the glenoid component. The glenoid prosthesis further can include various attachment systems known in the art for securing the tray to the anchor.

In various embodiments, the various dimensions and/or other features of the anchor or stem can be adapted to a particular patient's anatomy. For example, modeling of the scapular canal dimensions can desirably drive the subsequent design and/or selection of an appropriate scapular anchor. If desired, the scapular canal model can be queried or otherwise utilized to determine acceptable and/or recommended amounts of scapular anchor dimensions, curvature and/or tapering (as well as the location(s) of such tapering), which facilitates creation of an anchor that desirably remains within the canal during and after insertion. In various embodiments, the anchor design can be altered to accommodate and/or conform to a narrowing or widening (or other feature) of the canal. In a similar manner, the diameter of the anchor at one or more locations can be altered to conform to (or otherwise accommodate) diameter variations within the canal. In various embodiments, the anchor can include projections (e.g., flutes, barbs, threads, etc.) to further secure the stem within the canal. If desired, the anchor could include one or more threaded sections, such as in the form of a screw, which align with and are threaded into the canal wall. In one exemplary embodiment, the anchor could include proximal screw threads that secure the glenoid tray to the anchor. Of course, a wide variety of attachment mechanisms, including pins, pegs, screws, etc., could be employed to secure portions of the glenoid prosthesis to each other, as well as to the surrounding bone of the scapula. A variety of such attachment techniques can be employed, including the use of parallel-oriented Steinman pins (which can allow removal and/or replacement of tools from the surgical site while the pins remain placed within the bone) or non-parallel pins or holes at different inclinations (which can ensure secure and immovable fixation for a variety of reasons) or other fixation devices.

If desired, the anchor diameter could be sized slightly larger than the canal diameter in one or more dimensions to facilitate a “press-fit” type of fixation of the anchor within the canal. In alternative embodiments, the anchor could include an eccentric or “oval” shaped section, which desirably passes through an oval or irregularly-shaped restriction of the canal when the anchor is in one orientation, but subsequent rotation or other manipulation of the anchor prevents withdrawal of the anchor from the canal (e.g., it becomes “wedged” or otherwise cannot be removed beyond a certain predetermined restriction).

In various other embodiments, the canal modeling can be utilized to select and/or confirm selection of a pre-manufactured anchor that is appropriate to the patient's anatomy. If desired, additional steps can include selection of an anchor “blank” having dimensions and/or shapes proximate to the canal model, and then subsequent modification of the blank anchor can be accomplished (e.g., material removed and/or added, as appropriate) to particularize the anchor for the patient's scapular canal.

In addition to the design and/or selection of an appropriate scapular anchor and/or anchor blank, the canal modeling (as well as other patient-specific data and/or patient-adapted models) can be utilized to design and/or select appropriate surgical procedural steps and surgical preparation of the glenoid surface of the scapula as well as reaming of the scapular canal. The creation of patient-specific and/or patient-adapted surgical cutting and reaming tools, and associated guide tools, can significantly facilitate the accuracy and outcomes of a TSA procedure. The use of fluoroscopic, MRI or other actual images of body parts can facilitate the modeling and/or construction of surgical instruments and/or the position and orientation of body parts. Various anatomical information can be derived and utilized in the assessment of the anatomical structures, as well as the planning of the surgical procedure and associated implants/tools. For example, resection planes, anatomical axes, mechanical axes, anterior/posterior reference planes, medial/lateral reference planes, rotational axes or any other navigational or kinematic references or information can be useful or desired in planning or executing surgery.

In at least one exemplary embodiment, implants, tools and surgical methods are disclosed for performing shoulder arthroplasty, which can include imaging a patient's shoulder region and utilizing the anatomical image data to create a surgical plan for preparing the glenoid region of the scapula for an implant component, as well as planning the surgical access to and reaming of a canal in a lateral border of the scapula, further preparing the glenoid region to accommodate a glenoid tray component of a glenoid prosthesis (configured for articulation relative to a natural or prosthetic humeral head), and providing a scapular anchor configured to extend from an inner surface of a glenoid component into the scapular canal and configured to engage one or more structures within the canal for anchoring the glenoid prosthesis to the scapula. The various components of the shoulder prosthesis (which can be used in a total shoulder arthroplasty as well as replacement of one or more portions of the joint) can include a humerus prosthesis assembly and a glenoid prosthesis assembly. The glenoid assembly can include a glenoid component and a scapular anchor or stem. The humerus prosthesis assembly can include a stem or anchor, such as a humeral stem, and a humeral head that mates with the humeral stem and articulates in relation to and against an articulating surface of the glenoid assembly. A variety of anchoring techniques for the glenoid and humeral prosthesis can be contemplated, including pins, stems, anchors, pegs, screws, adhesives and/or other known means for anchoring an implant to an underlying bony support structure. Preferably, the humerus and/or glenoid prostheses can include features that approximate the general shape of the natural humerus and/or natural glenoid/scapula, though other shapes that mate with an opposing surface (e.g., glenoid and/or humeral articulating surfaces) may be contemplated. When replacement of the humeral head is not indicated or desired, a partial joint replacement, such as resurfacing or replacement of the glenoid surface alone, can be employed, with the glenoid component designed, selected and/or modified to mate with the natural humeral head. In a similar manner, various approaches and techniques may be employed where only the humerus requires resurfacing/replacement, and the glenoid cavity and/or glenoid articulating surface(s) remains substantially intact.

In various embodiments, implant components, guide tools and surgical procedural steps can be designed/selected and/or modeled to accommodate and/or facilitate a specific type and/or orientation of surgical access procedure. For example, where an anterior surgical access path is contemplated, it may be desirous to design and/or select implant components and surgical tools to easily pass through the surgical incision(s), and be placed in the targeted anatomy within the anticipated readily-available surgical volume. In one example, a scapular anchor design may be modified depending upon the intended surgical access path, with a superior access to the shoulder allowing for a longer, straighter scapular anchor (which accommodates the patient-specific anatomy) while an anterior access path may mandate or prefer a shorter, more curved scapular anchor (which can be rotated and/or otherwise manipulated within the surgical volume as it is inserted within some portion of the scapular canal). Similarly, guide tools may align with various anatomical features that are directly exposed along a preferred access path, while other anatomical features may still be masked by overlying tissues.

Various embodiments described herein include the use of patient-specific anatomical data in planning and/or executing less-invasive or minimally invasive procedures to access the articulation region and the joint capsule surrounding the humeral head and the glenoid, to allow for replacement of at least one of (or portions thereof) the glenoid or the humeral head. The procedure can be performed by accessing the rotator cuff capsule by an incision near the shoulder and separating various muscle and/or tissue bundles and then incising the capsule. The procedure may be performed without substantial removal or resection of the subscapularis muscle or its attachment near the glenohumeral joint. Also, other muscles forming the rotator cuff can remain intact as well. As described herein, a prosthetic can be designed, selected and/or modified to facilitate insertion and placement within the incisions, which in various embodiments can include assembly of some or all components within the incision.

In at least one exemplary embodiment, a method of performing an arthroplasty on at least one of a glenoid or a humeral head of a humerus through soft tissue anatomy is disclosed. An incision can be formed in the soft tissue near a superior-lateral portion of the glenohumeral joint and portions of the deltoid muscle are separated substantially superior and lateral of the glenohumeral joint. The humeral head can be resected and a prosthetic stem can be inserted into the intramedullary canal. After insertion, a humeral head component can be positioned onto the stem to replace the resected humeral head. At various points in the procedure (such as where the native humeral head has been resected and removed), the glenoid and/or a scapular canal can be prepared, and a patient-specific and/or patient-adapted scapular anchor can be inserted into the scapular canal and a glenoid implant component inserted into the prepared glenoid and secured to the anchor. The separated muscle tissue and the incision in the soft tissue can be closed. The rotator cuff muscles, including the subscapularis muscle, can remain substantially or completely connected during the arthroplasty procedure.

In various embodiments, prosthetic components are provided that allow for ease of accessing the anatomical portions and performing the less invasive procedure. For example, stem and anchor designs and configurations including fixation mechanisms (or other features) that allow a superior approach to implant the stem/anchor (via the incision) can be provided that interconnect with selected portions of the implant components. In various embodiments, glenoid components and/or humeral head components having coupling members or other attachment mechanisms and/or arrangements with a central axis that are not perpendicular to a glenoid/head interface surface can be used in the afore mentioned approach. Various prosthetic insertion methods (including the use of differing approaches from different angles and/or directions) are contemplated using prosthetic components (as well as other designs) to achieve the desired TSA.

Various instruments can be used in performing a selected procedure, such as a total shoulder arthroplasty procedure, such as the replacement of a humeral head and a glenoid where the humeral head and the glenoid can articulate with one another after implantation. In various alternative embodiments, various similar instruments and procedures may be used to perform a hemi-arthroplasty, such as replacement of only one (or portions thereof) of a humeral head or a glenoid.

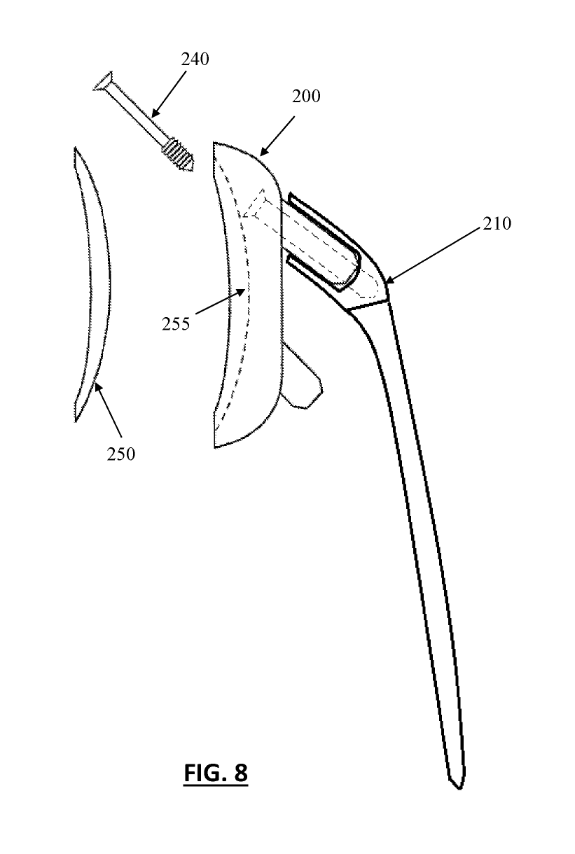

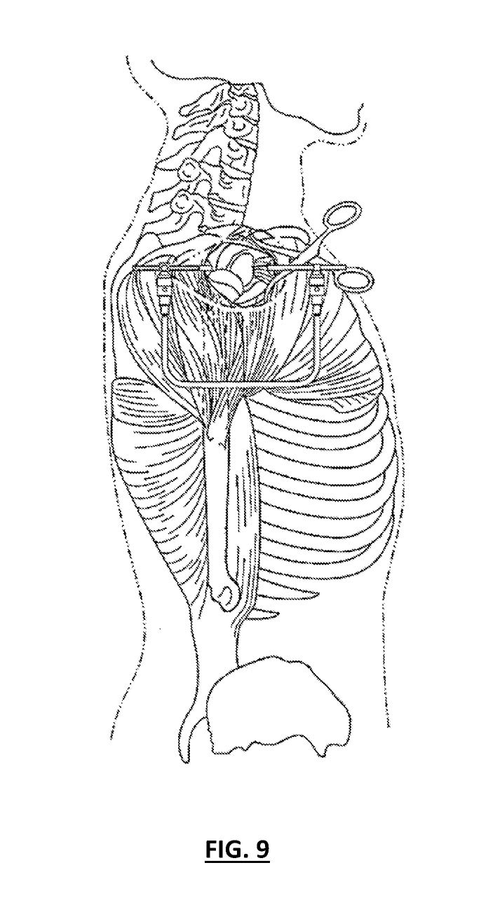

FIG. 9 depicts a partial side view of a human torso, with various subcutaneous layers exposed, and a shoulder region being accessed through an external skin layer and soft tissue below, such as muscle. Various portions of the anatomy, including the humerus and the glenoid region of a scapula, can be accessed by forming an incision in the soft tissue, including the skin. To access the shoulder joint, various subdermal portions, such as subdermal adipose tissue, can be incised along an incision. It should be understood that an incision can be orientated in virtually any appropriate direction such as anterior to posterior, which is generally parallel to a sagittal plane. In at least one exemplary embodiment, the incision can be about 5 cm in length. Various other alternative incision approaches, such as a superior-inferior incision which is generally along the coronal plane, can be made.

The skin incision can be made parallel with Langerhan's lines at the superior aspect of the shoulder, just even with the lateral border of the acromion. The incision can also be medialized slightly, if desired. The incision can be any appropriate length, and may depend upon surgeon preference, patient type, prosthetics to be used, or other indications, as well as the location and condition of various tissues and other anatomy that may be visualized and/or modeled using patient anatomical image data, as described herein. In various embodiments, the incision can be from approximately 3 cm to approximately 20 cm in length, and in one exemplary embodiment can be approximately 7.5 cm to approximately 10 cm.

Depending upon surgeon preference and training, the incision through the skin may be shorter than the area opened in the muscle. The incision can be used to achieve access to the muscle that is around the various portions of the anatomy selected to be resected, including the humerus and the glenoid surface of the scapula. Desirably, the incision can permit access to a deltoid muscle.

A retractor, such as a Gelpi Style Retractor, can be used to retract soft tissues in a known manner, such as the muscles surrounding the glenohumeral joint (including the deltoid muscle). If desired, the retractor may be employed to expand the incision to gain access to the muscle. The retractor, as illustrated herein, can be virtually any surgical tool used to retract or position the deep tissue that is generally near the glenohumeral joints.

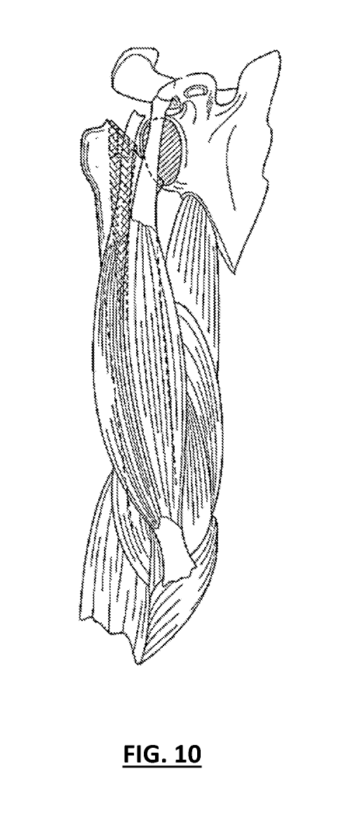

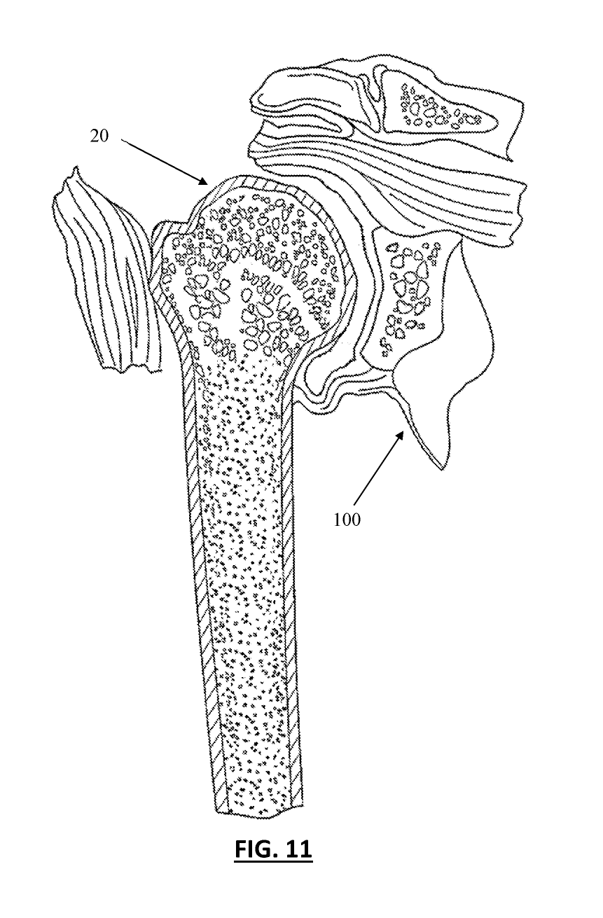

In one exemplary embodiment, a passage can be formed via an incision through the deltoid to access various deeper soft tissue portions, such as the sub-deltoid bursa and the subacromial bursa, without damaging the rotator cuff. Depending upon surgeon preference, various other deep soft tissue can be incised and/or moved to facilitate access to the capsule surrounding the shoulder (or glenohumeral joint). After moving and/or incising various tissues and/or portions, access to the humeral head and glenoid portion of the scapula can be achieved (see FIGS. 10 and 11).

Various surgical tools such as retractors can be used to hold the various soft tissue portions open, such as the cuff interval, capsule and the like. Various soft tissues adjacent the capsule may be incised and/or resected, as desired. For example, the bicep tendon interconnecting at or near the humeral head may be resected or may be moved, if already detached, to achieve better access to the humeral head. In various embodiments, access to the glenoid surface of the scapula can be facilitated by the incision and/or removal of various soft tissues.

When employing a surgical approach to the shoulder joint capsule via a shoulder incision near the glenohumeral joint, such as disclosed in one exemplary embodiment, various muscles and ligaments can be retained and maintained substantially intact during glenohumeral joint access. For example, the subscapularis muscle and the ligaments attaching it to the portions of the glenohumeral joint need not be incised, if desired. The subscapularis muscle can be left intact, as it is generally anterior from the disclosed approach. If desired, the supraspinatus can be left and/or remain intact, as can all the muscles of the rotator cuff. This approach allows the passage to be formed by separating the cuff interval rather than detaching or incising various soft tissue portions. Moreover, the humeral head need not be substantially dislocated or dislocated at all from the glenohumeral joint. Rather, the humeral head can be moved or otherwise distracted or displaced to allow access to various portions of the anatomy, including being left in its typical anatomical position and/or retracted any appropriate distance, such as about 2 cm to about 8 cm.

To complete access to the glenohumeral joint, the soft tissue over the biceps laterally can be sharply dissected off the humerus down to the top of the subscapularis tendon, with the tendon left substantially undisturbed. The supraspinatus may be stripped back off the anterior portion of the greater tuberosity for a distance of about 5 mm to about 10 mm to further enhance the exposure. If desired, no less than about 1 cm of the tendon could remain attached, retaining the basic integrity of the tendon. This exemplary exposure of the rotator interval can give an approximately 1.5 cm to about 2 cm gap at the lateral edge, without disrupting the rotator cuff mechanism. If desired, the retractor can be moved from the deltoid to the rotator interval to provide greater exposure of the glenohumeral joint.

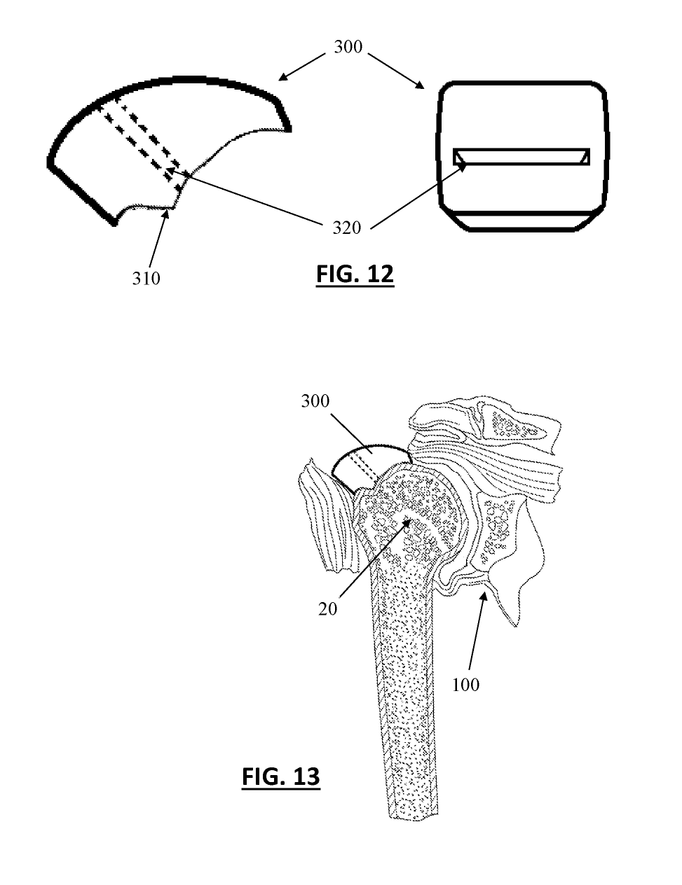

Once the glenohumeral joint has been accessed, such as shown in FIG. 11, various instruments can be employed to prepare the relevant anatomical structures for receiving implant components and associated anchoring and/or fixation devices. For example, FIG. 12 depicts side and front views of a guide tool 300 designed using patient-specific image data to include a surface 310 that matches or substantially conforms to a surface of the humerus accessible through the incision, such as shown in FIG. 13. In this embodiment, the patient-specific surface (which is now easily accessed through the pre-planned approach) can match a portion of the humeral head contour that was previously visualized and/or modeled, which may include and/or accommodate the presence of osteophytes, voids and/or other irregular features on or adjacent to the humeral articulating surface. The guide tool 300 can further include a surface or slot 320 that is sized and configured such that a surgical tool can pass through the slot 320 and access the humeral head to cut, drill, ream or perform other surgical procedural steps on the humeral head or other aligned anatomy. Desirably, the conforming surface 310 of the tool will align with matching features on the humeral head, thereby aligning the slot 320 relative to the humeral head and allowing precise surgical resection and/or other preparation of the humeral head, as desired. In use, the physician typically holds the guide tool (or a handle, if provided) with one or more hands and presses the tool against the joint surface. Tactile and visual clues desirably resulting from the conforming/matching surface(s) allow and facilitate registration of the instrument body with the native anatomy.

In various alternative embodiments, and depending upon the amount of humeral anatomy exposed during the surgical procedure, the humeral guide tool can comprise a “cap-like structure” that can be connected to an offset “block” feature, such as an offset block that contains a saw capture guide for resection of the entire humeral head (which is concurrently being used to guide and/or align the cap-like structure). If desired, a clearance volume between the “cap” and “block” can be provided (or other linkages or arrangements, including removable features and/or adjustable features, as desired). The cap can have an inner surface and an outer surface. In this respect, the engagement surface can be generally concave, but can also include convex portions corresponding to concave portions of the head. Although the outer surface of the cap can have any shape, for a thin stretchable cap the outer surface can be generally convex or semi-spherical. In one embodiment, the cap can terminate at or about the anatomic neck.

In various embodiments, the humeral guide tool can include one or more alignment pin openings or other feature that accommodate the placement of reference pins for guiding other instruments or templates or sizers, and potentially be used as a securing device during resection. The opening(s) or other feature(s) can be in the form of one or more elongated tubular elements (or other shapes) extending from the cap and having an elongated open ended guiding bore. The guiding bore can be designed during the pre-operative plan with input from the surgeon such that an alignment pin can be guided by the bore to a predetermined location on the resected surface of the humerus, either centrally or at some offset and at a patient-specific orientation, either perpendicularly or at an angle to a planned resected surface. The alignment pin can be driven through the cancellous bone of the humerus all the way through the lateral cortex to help secure the alignment pin. In some procedures, the alignment pin can also be used to guide a separate cutting guide (or other tool) for resecting the head after the patient-specific guide is removed.

In one exemplary embodiment, a patient-specific humeral head guide tool and/or implant components can be designed and/or selected using MRI or CT data to determine the appropriate orientation and size of the orthopedic component. For shoulder hemiarthroplasty and/or total shoulder arthroplasty, the position of the humeral component could be approximately 20 degrees in retroversion. If desired, a MRI or CT scan of the elbow from the same side of the body can be used to properly correct the version of the humeral head. If desired, the diaphysis of the humerus could be approximated to be a cylinder, with a long axis defined as the long axis of the humerus. Landmark points could be placed on the medial and lateral epicondyles of the distal humerus. A humeral coronal plane could be constructed that passes through the landmark points and is parallel to the long axis. The version of the humeral head could be offset from the coronal plane. If the elbow has not been scanned or otherwise imaged, the calcar of the humerus can be used as a reference when determining version angle, and a calcar landmark point identified. In such a case, the version plane of the humeral component can be defined as the plane that passes through the calcar point and the long axis of the humerus.

In one embodiment, the level of resection of the humeral bone can be built into the humeral head guide tool and/or cutting block. Using MRI or CT data, the guide tool will desirably engage with the humeral head by having a backside face that is a 3D inverse of one or more portions of the native humeral head, using a model of the anatomical image data created using a Boolean subtraction operation where the native surface of the humeral head is subtracted from a template block instrument. Where desired, an approximately 1 mm gap between the bony surface of the humeral head and the inverse surface of the humeral head cutting block can be added when using CT data to accommodate cartilage and/or slight errors in the reconstruction. Alternatively, a cartilage coring operation and associated coring guide, with an associated guide tool including offset subchondral bone reference pegs, could be utilized. Employing such designs, the block can desirably engage the superior-medial aspect of the head, and may have one or more additional features that wrap around the lateral side of the lesser tubercle (such as a subscapularis attachment sight) to additionally aid in the alignment of the tool. The instrument can include one or more openings to allow the subscapularis and rotator cuff to pass without impingement. One or more slots for saw blades can be located approximately anterior to the humerus, with a pre-defined cutting angle (for example, approximately 45 degrees) being predesigned or otherwise integrated into the designed or selected/modified implant system. In various embodiments, the slot can have sufficient width to ensure that the blade remains substantially parallel to the slot during the resection operation.

Other features of an exemplary humeral guide tool could include two or more non-parallel pin holes for additional stability of the block connection to the proximal humerus, or two or more parallel pin holes that may facilitate removal of the guide tool and replacement with a subsequent guide tool, jig or other instrument (including an instrument to align glenoid/scapular tools). In various embodiments, pin holes can be located distal to the saw blade slot, and can accept pins, screws or other fasteners. If desired, viewing slots or other portals on the tool can be provide to allow the surgeon to visually ensure that the instrument is fully seated onto the humeral head. A targeting sight in line with the long axis of the humerus on the superior surface of the humeral head guide tool could be used to target a humeral stem reamer.

In various embodiments, it may be desirous to additionally ream the humeral canal in preparation for implantation of a humeral stem. In one exemplary embodiment, a humeral reamer (which can be patient-specific, patient-adapted and/or a standard reaming tool) can be reamed into the humerus near the humeral head. Humeral reaming can occur from the superior, lateral humeral head. The entrance to the head can be just underneath the natural location of the biceps tendon. The arm can be extended slightly, and the elbow can be placed against the patient's side to bring the top of the humeral head forward, and allow the reamer to pass the front of the acromion. This approach and technique can allow the humeral head to be retracted in a known manner, but remain substantially or completely undislocated, which can reduce trauma in the surrounding soft tissue. The superior approach allows easy centering of the reamer in the humeral head and proximal shaft, and decrease the initial incidence of varus stem placement and/or eccentric head utilization.

If desired, one or more patient-specific and/or patient-adapted stems can be configured to be implanted into the prepared humeral medullary canal prior to the coupling of the stem to a humeral head. The selected stem (or the single stem, if only one patient-specific and/or patient-adapted stem is provided) can be implanted into the canal by applying impact forces along a central axis in a known manner. The impact direction can be independent of the angle of the head coupling surface, if desired. In various embodiments, the stem can be configured to accept a variety of humeral head shapes, sizes and/or orientations after the stem has been implanted into the patient. In this manner, the disclosed design (and associated surgical approach) can allow a significant reduction in the size of the needed incision in the subscapularis muscle. In at least one exemplary embodiment, the humeral head and stem include a coupling mechanism, such as a male and female locking taper configuration, as well known in the art. In various alternative embodiments, the humeral head can be coupled to the humeral stem via an intermediate coupling member, which may include a variety of such members of varying configurations, if desired.

In various embodiments, the humeral reamer can comprise a shaft or other feature that can extend from the humerus. The reamer can be positioned into the humerus and be interconnected with various portions, such as a patient-specific and/or patient-adapted guide tool or jig. The guide tool can integrate with the shaft of the reamer, with the reamer still within the humerus, and the guide tool can be used to align desired tools and/or be utilized as an interconnection and/or other feature to align cutting or preparation tools relative to the humerus, the humeral head and/or other anatomical features of the shoulder joint (e.g., the glenoid space and/or scapular canal).

In at least one embodiment, a patient-specific jig can be used to orient a cutting guide in a proper and/or desired orientation relative to the humeral head or other anatomical feature of the humerus and/or shoulder. A jig can be used to obtain or position an axis of the cutting guide, such as a central axis, relatively in line with the humerus. This arrangement can help position the guide surface generally perpendicular to an axis of the humeral head, if desired. The axis can be generally perpendicular to a plane or line extending through the humeral head and/or through the elbow or other anatomical feature remote from the shoulder.

In various alternative embodiments, the jig can align a cutting guide to position and/or align (or otherwise provide and/or define) a cutting tool or instrument at approximately 20 to 30 degrees of retroversion. Once the jig provides this desired alignment, the cutting tool and/or jig (or components thereof) may be held in place with a fixation pin or other arrangement, desirably allowing removal of the reamer or other alignment devices for subsequent resection of the humeral head. In various embodiments, the guide tool or jig can be held in place (including the use of a pin or other fixation mechanism) when all the other portions of the apparatus are removed. A saw can then be used to resect the humeral head, with the blade riding along a portion of the guide tool. The guide tool can desirably ensure a proper orientation and/or position of the saw blade relative to the humeral head. Further, a glenoid shield (or various portions of the guide, include a guide thickness and/or other arrangements) can be positioned relative to the glenoid and other portions of the anatomy (if desired) to assist in ensuring that the saw does not engage portions of the anatomy not desired to be cut.

It should also be understood that various cut planes and/or other surgical preparation of various anatomical structures, including the humeral head, can be begun with a guide tool or jig, and then finished without the guide tool or jig. For example, an initial portion of the humeral head can be resected with use of a cutting guide tool. After an initial portion of the cut is formed the cutting guide tool and any fixation pins can optionally also be removed. The remainder of the cut of the humeral head can then be performed using the initial portion of the cut formed with the saw blade to guide the remaining portion of the cut. In various embodiments, therefore, the cutting guide tools (and/or other alignment features, including canal reamers) need not be present during the entire cutting operation to form the entire cut, notch, drill hole or reamed structure or other preparation of a given anatomical feature, such as a humeral head.

Once the humeral head has been resected to a desired amount (or where surgical access to the glenoid is otherwise facilitated prior to or without such humeral resection, if desired, such as by retracting the humeral head away from the glenoid surface of the scapula), the glenoid surface and associated scapular structures can be prepared in a similar manner. The glenoid condition can also be assessed, and a decision can be made for hemiarthroplasty or total shoulder arthroplasty. Where the glenoid is well visualized, and directly approached as described herein, the surgical exposure can be lateral as compared to other techniques. Glenoid version, glenoid erosions, and glenoid osteophytes can be easily assessed and removed or modified, if desired. Labral tissue can be cleared from around the margins, and glenoid preparation can be carried out with a selection of guide tools and instruments. While various embodiments herein describe humeral then glenoid preparation, glenoid preparation and/or implantation can occur prior to humeral broaching and/or preparation/implantation. It should also be understood that the glenoid may alternatively be first prepared (before the humerus or any other anatomical structures) using various techniques and/or procedures described herein.

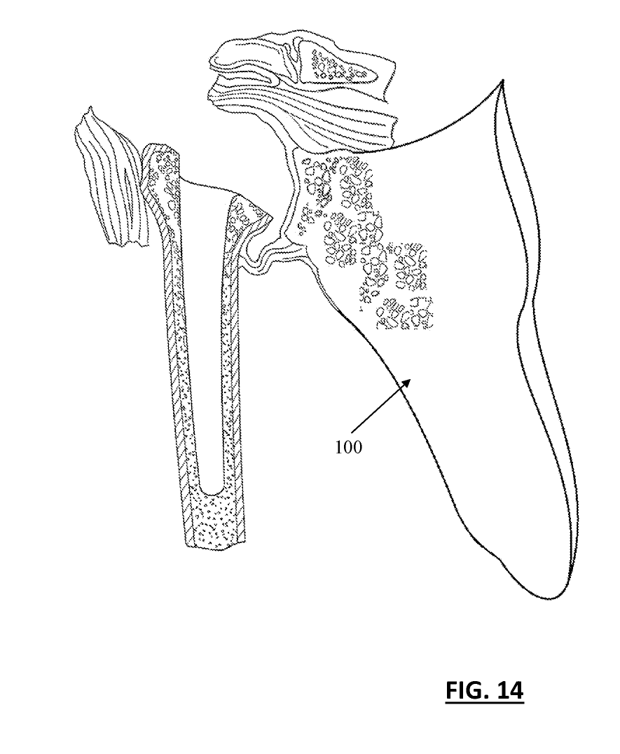

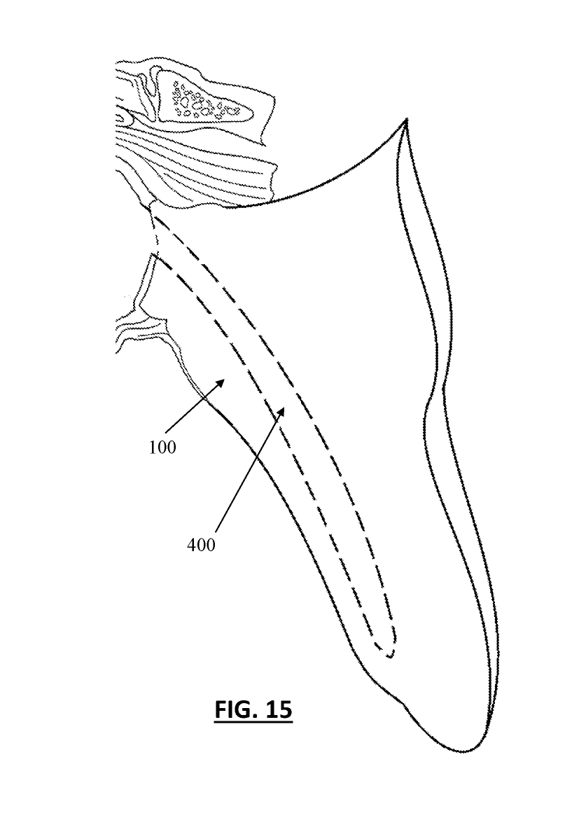

FIG. 14 depicts a view of a shoulder joint incision including a resected humeral head (and prepared humeral intramedullary canal) and a partial cross-sectional view of the glenoid cavity and relevant portions of the scapula. Using similar guide tools as previously described to align surgical tools relative to the glenoid and/or scapula (using, for example, patient-specific and/or patient-adapted anatomical information and/or models to create surgical tools and guide tools), a reamer can be reamed into the glenoid cavity proximal the scapular neck and into a relatively thickened portion of the scapula proximal the lateral margin. In various embodiments, a patient-specific reamer and/or other surgical tools can be designed/selected and utilized to create a canal 400 and/or channel within the relevant scapular section, such as shown in FIG. 15.

If desired, some or all of the glenoid cavity can be reamed prior to preparation of the scapula canal. For example, various guides, including those described herein, can be used to assist in achieving these procedures. As discussed herein, various connecting portions or other arrangements can be employed that use patient-specific and/or patient adapted guide tools and/or jigs to position tools or other devices at a desired location and/or orientation of the glenoid surface. In one exemplary embodiment, a reamer can be connected to a reamer shaft and a power source such as a drill or reciprocating saw. If desired, the reamer shaft can include a flexible or other portion (e.g., angled rotatable coupling) that allows for deformation of the reamer shaft. The guide too or jig can be used to align the reamer and control the angulation, orientation and/or depth of reaming/cutting of the glenoid cavity and/or scapular canal. Various embodiments and arrangements allow the reamer to be rotated and/or advanced/retracted relative to the glenoid and/or the drill or other power tool in a desired manner to form the glenoid cavity into a selected shape and orientation. The glenoid may be shaped to allow for implantation of a selected glenoid implant. It should be understood, however, that the glenoid need not necessarily be resected or otherwise shaped, and a glenoid tray component that conforms to some or all the pre-existing anatomical features (and desirably connects to a scapular anchor or other fixation arrangement) and articulates with an implant positioned in the resected humerus is contemplated.

If desired, a glenoid guide tool or jig can be employed in a similar manner to the humeral tools to align relative to the glenoid surface, the humerus and/or within the distracted joint (e.g., against both the glenoid and humerus, as well as against or in relation to any other individual or combination of exposed surfaces and/or implant structures, such as a surface of a humeral stem) and facilitate the creation of a scapular canal. The glenoid reamer may be navigated to determine the depth, position and angle of reaming. Subsequently, other glenoid instruments may be used to prepare the glenoid to receive a glenoid component and/or component trial. Any appropriate glenoid component or component trial may be used, for example, an all-polyethylene glenoid component with three pegs or one keel or with a metal back. Such glenoid components can include one or more screw holes or other fixation augments on the glenoid base. Depending on the type of glenoid component used, a drill guide or keel reamer guide may be used to prepare the glenoid for the glenoid component. In one exemplary embodiment, a first glenoid jig is utilized to create a patient-specific scapular canal, and when complete a patient-specific scapular anchor is inserted and positioned within the canal. If desired, a second glenoid jig can then positioned over and in a predetermined alignment with some portion of the implanted scapular anchor (such as, for example, over an exposed proximal end of the scapular anchor within the joint space), and various features of the glenoid jig can be utilized to prepare the glenoid space for a patient-specific and/or patient-adapted glenoid tray component. Once the glenoid space is properly prepared using this second jig, the glenoid jig can be removed and the glenoid tray component is implanted within the prepared glenoid space and secured or otherwise fixed to the scapular anchor. In various alternative embodiments, the glenoid space may be prepared first, and then a jig used in the glenoid space to subsequently guide the preparation of the scapular canal.

In at least one exemplary embodiment, a glenoid guide tool can include a generally oval or circular body with an attached handle. The body can include one or more patient-specific surfaces that conform to and/or substantially match one or more surfaces of the existing glenoid and/or scapular structure, which may include one or more articular surfaces, subchondral bone surfaces, soft tissue structures and/or artificially-created surfaces (e.g., previous cut planes and/or pre-existing joint structures created during the current and/or during a previous surgery now being revised). The body may also include one or more surfaces that conform to and/or substantially match one or more surfaces of adjacent anatomical structures and/or implant components, such as the humerus or a humeral stem/head. Adjacent the patient specific surface(s) are features that match other articular bony portions of the glenoid or scapula, which can include one or more hooks or projections formed depending on the patient anatomy. Such features can be distributed in various portions of the body to accommodate, align and/or designate various surrounding anatomical structures (e.g., tendon attachment points). If desired, the body can further include one or more holes or slots, passing through the instrument body, which desirably extend from a lower surface to an upper surface. Such holes or slots can be useful for a variety of reasons, including to direct and/or align cutting instruments, drilling instrument, reaming instruments, to visualize native surfaces through the holes and/or to be used for the placement of alignment and/or securement pins. Holes can also be useful for aligning of coring or debriding instruments for removal of specific locations of articular cartilage on the glenoid/scapular surface, exposing one or more subchondral bone surfaces that can subsequently be used to align further guide tool instruments. The use of subchondral bone alignment in this manner facilitates the alignment of subsequent tools, as subchondral bone is generally easier to visualize than articular cartilage and/or other soft tissues, thus providing a more reliable reference surface for the surgical procedure.

If desired, the glenoid guide tool can include one or more windows to permit visual confirmation of placement. The tool may also include a handle or other feature to assist in proper positioning of the instrument. If desired, the tool can include a variety of holes or other features that allow the surgeon a plurality of options in defining the direction of screws or other fixation features, should screw placement be pre-operatively determined or where the need for screw fixation (or additional unexpected need for fixation) becomes apparent during the surgical procedure.

In various embodiments, a subsequent glenoid guide tool or jig can be positioned relative to the reamed glenoid (and is desirably sized and/or shaped to accommodate the modified anatomy), and include various features such as openings for drilling or forming a plurality of bores in the resected glenoid surface with a drill or bit interconnected to a drill motor or other surgical device. Using such a patient-specific and/or patient-adapted guide tool, various bores can be formed in the resected glenoid surface to allow for securement and/or positioning of portions of the glenoid tray, including pegs or stems extending from the bone-facing surface of the tray into the glenoid/scapula. The pegs can be employed to resist a variety of tray motions, such as rotation, translation, subsidence/depression, surface separation and the like. Further, the pegs can allow for cementation points to cement the glenoid implant to the glenoid cavity, if desired. The locations, sizes and orientations of the pegs, and the cavities to accommodate such pegs, can be designed and/or selected using patient-specific and/or patient-adapted models such that the cavities are appropriate to the patient's scapular anatomy and their presence does not significantly reduce the strength of the native bone structures or endanger soft tissue attachments thereto. The various techniques described herein can include evaluation of the “fit” of a glenoid keel or pegs within the glenoid space (and/or other scapular anatomy) during design/selection of the implant, tools and cut guides, as well as before bone preparation is performed, to insure that “breakthrough” or other damage to the posterior aspect of the scapula does not occur.

In various alternative embodiments, a reamer or other surgical tool can be used to initiate and/or create some or all of the scapular canal, and then a glenoid guide tool or jig may subsequently integrate with the reamer (or other tool) while still within the canal to align one or more tools to prepare and/or align the glenoid cavity for the glenoid tray. In one exemplary embodiment, a “starter tool” can be used to create some portion of the scapular canal, and then the starter tool can be used, at least partially, to align one or more tools to create and prepare the glenoid cavity, and then (if desired) a further tool can use the prepared glenoid cavity to align a subsequent surgical tool for preparation of the completed canal. Such an arrangement can facilitate initial identification and alignment relative to a centroid (or other desired alignment) of the glenoid surface and/or other anatomic feature (e.g., an axis of the scapular canal), and then final alignment of the canal can be accomplished after preparation and/or implantation into the glenoid cavity.

In various embodiments, the employment of patient-specific and/or patient-adapted reamers and surgical guide tools for preparing the scapular canal and/or the glenoid surface can significantly reduce surgical errors and/or potential complications. Unlike more regular long bones such as the humerus, the femur or the tibia, the scapula (and the scapular canal) is typically an irregularly shaped plate-like bone, with significant structural variation among the healthy population. In a typical shoulder joint replacement procedure, much of the scapula is not exposed, and thus there is little or no opportunity for a surgeon to directly visualize a violation or fracture of the scapula or scapular surface below the expose glenoid surface. Such fractures can significantly affect the integrity of the scapula and/or shoulder, as well as allow fixation materials (such as bone cement) to exit the scapula and impinge upon other tissues and/or enter the vasculature. Moreover, surgical tools that exit the scapula in an unintended manner during the surgery (such as through a fracture) can cause significant damage to many important anatomical structures adjacent the scapula, including major blood vessels and/or nerve complexes. By utilizing patient-specific image data (and modeling thereof), and creating implants, tools and surgical techniques appropriate to the imaged/modeled anatomy, the surgical procedure, and the ultimate fixation of the implant components, can be significantly improved.

Various features described herein can also include the use of patient-specific and/or patient-adapted image data and models to determine the opportunity, incidence, likelihood and/or danger of unintended and/or accidental damage to adjacent anatomical structures. Depending upon the surgical repair and the physician's preference, various anatomical structures such as nerves and/or major blood vessels may be preferably avoided, which may alter the ultimate surgical procedure and/or guide tools, instruments and/or implant components designed, selected and used to accomplish a desired surgical correction. The use of such data to ensure clearance spaces, accommodate blocking structures (e.g., reamers or shields to protect various areas from cutting instruments) and/or to modify guide tool alignment and/or structures is contemplated herein. For example, a humeral guide tool could include a clearance space or solid projection that avoids or shields muscle and other tissue, thereby minimizing opportunity for inadvertent injury.

Implant design and modeling also can be used to achieve ligament sparing, for example, with regard to the subscapularis tendon or a biceps tendon. An imaging test can be utilized to identify, for example, the origin and/or the insertion of the subscapularis tendon or a biceps tendon on the glenoid/scapula. The origin and the insertion can be identified by visualizing, for example, the ligaments directly, as is possible with MRI or spiral CT arthrography, or by visualizing bony landmarks known to be the origin or insertion of the ligament such as the medial and lateral tibial spines and inferring the soft tissue location(s). An implant system can then be selected or designed based on the direct or inferred image and location data so that, for example, the glenoid component preserves the subscapularis tendon or a biceps tendon origin. The implant can be selected or designed so that bone cuts adjacent to the subscapularis tendon or a biceps tendon attachment or origin do not weaken the bone to induce a potential fracture.

If desired, the glenoid implant can have a plurality of unicompartmental articulating surface components that can be selected or designed and placed using the image data. Alternatively, the implant can have an anterior or posterior bridge component or other connection feature between multiple surface components.

Where the glenoid implant includes one or more insert components, the margin of an implant component, e.g. a polyethylene- or metal-backed tray with polyethylene inserts, can be selected and/or designed using the imaging data or shapes derived from the imaging data so that the implant component will not interfere with and stay clear of the subscapularis tendon or a biceps tendon. This can be achieved, for example, by including concavities and/or voids in the outline of the implant that are specifically designed or selected or adapted to avoid the ligament insertion.

Any implant component can be selected and/or adapted in shape so that it stays clear of important ligament structures. Imaging data can help identify or derive shape or location information on such ligamentous structures. For example, an implant system can include a concavity or divot to avoid the tendon or other soft tissue structure. Imaging data can be used to design a component (all polyethylene or other plastic material or metal backed) that avoids the attachment of the various tendons/ligaments; specifically, the contour of the implant can be shaped so that it will stay clear of such structures. A safety margin, e.g. 2 mm or 3 mm or 5 mm or 7 mm or 10 mm can be applied to the design of the edge of the component to allow the surgeon more intraoperative flexibility.

In various embodiments, a length, diameter and shape (as well as other features) of the anchor can correspond to a length and diameter of the canal (or portions thereof), with the canal dimensions previously obtained and/or planned using patient-specific anatomical data, as described herein. Further, the angle formed between the anchor and the glenoid tray can correspond to an angle between the canal and the natural glenoid of the shoulder, which may also be predetermined using patient-specific anatomical data. In at least one exemplary embodiment, the scapular anchor can comprise a generally curved, frustoconical shape, which can initially extend perpendicular or at an angle from a bone-facing side of a glenoid tray or other implant component, and then curve downward smoothly or at an acute or obtuse angle, with the anchor engaging a natural and/or artificially created canal in the lateral border of the scapula.

In various exemplary embodiments, a patient-adapted and/or patient-specific glenoid implant can be utilized, per the surgeon's preference and as discussed herein. FIGS. 6 and 7 depict rear and side views, respectively, of a glenoid prosthetic tray 200 configured to be used in various embodiments of a total shoulder arthroplasty procedure as described herein. The glenoid tray 200 includes a curved inner surface 255 and a generally flattened outer surface 257. The outer surface 257 is sized, shaped and configured to be coupled to a resected glenoid surface (not shown) and includes an engagement structure 220 (for engaging a scapular anchor, as previously described) and one or more coupling pegs 230. In various embodiments, the coupling pegs 230 can have a plurality of intersecting axis which are a predetermined angle from a plane defining the outer surface 257, the inner surface 255, one or more insert surfaces (not shown) or any combinations thereof. Alternatively, the angulation, shape, thickness and/or depth of pegs can be designed and/or optimized using patient-specific and/or patient adapted image data and/or modeling data, to ensure adequate bone quality for fixation as well as to minimize fracture and/or unwanted thinning of relevant bone structure of the scapular neck. In various exemplary embodiments, the angle could be between about 100 to about 60 degrees, and preferably between about 30 to about 45 degrees. If desired, the glenoid tray, inserts and associated fixation pegs can be configured to facilitate the insertion of the glenoid tray using a superior approach through an incision to the resected glenoid.

In various embodiments, including the embodiment depicted in FIG. 8, the glenoid tray can comprise a metallic base which includes a corresponding inner surface 255 for receiving a polymer or other material (e.g., plastic, metal and/or ceramic) insert 250. The tray (or other base member) can be coupled to the resected glenoid using stems, anchors or other devices, including bone coupling screws (as known in the art) as well as being secured or otherwise fixed to the scapular anchor 210. In one embodiment, the scapular anchor 210 can be secured to the tray 200 via a male/female “prong and socket” arrangement, with a supplemental screw 240 employed to fix the prong and socket together. In various embodiments, the various anchoring and/or attachment features (as well as any supplemental fixation structures for securing the glenoid tray to the surrounding scapular bone) can be angled and/or oriented in various manners, including parallel alignments that facilitate access through the superior approach and insertion of the tray into the prepared glenoid socket.

In various alternative embodiments, the glenoid tray can include an opening or other feature to accommodate some portion of the scapular anchor, with a dimension of the opening at an inner face being smaller than a corresponding dimension of the end of the scapular anchor, such that the anchor can be wedged within and/or otherwise secured into the opening. The end of the anchor can be threaded to mate with matching threads in the surface of the tray to secure the tray to the anchor. The end of the anchor can be flanged to engage a shoulder formed within an opening in the tray. As the anchor is further engaged into the canal, a force is exerted by the flange against the shoulder and can secure the tray to the anchor. In one exemplary embodiment, a bolt could be threaded on the end of the anchor, such that a head of the bolt could engage a portion of the tray, including portions of the shoulder and/or opening, as the bolt is threaded or otherwise engaged (e.g., a bayonet-type fitting engagement).

If desired, a glenoid tray or other similar component can be positioned on and/or fixated to a natural or prepared glenoid surface of the scapula, with some portion of the implant (or an insert component not yet implanted therein) desirably approximating an orientation of the natural glenoid. The tray can include an opening or other feature that is generally aligned or otherwise in a known orientation relative to a scapular canal when the tray is positioned on the scapula. If desired, the tray may be secured to the glenoid surface (prepared and/or natural) before the scapular anchor is subsequently inserted through the opening and into the canal. The anchor can include a wide variety of shapes, forms and sizes, including that of a screw which aligns with and can be threaded into the canal. The proximal end of the anchor can include an enlarged portion or flange, which can bear against the tray in a known manner as the screw is advanced into the canal, thereby further securing the glenoid tray to the underlying scapula. In various embodiments, the scapular canal can be prepared through the opening, after the glenoid tray (and/or a “trial” glenoid tray component) has been implanted.

Various configurations of the anchor are described and contemplated herein. If desired, the anchor can be threaded, fluted, and/or can have barbs extending outwardly from the outer surface for engaging the stem within the canal. The anchor can include moveable and/or deformable portions, including the use of shape-memory or martensitic materials, which can selectively engage surrounding tissues upon reaching a desired temperature and/or state. The anchor can include one or more longitudinal openings extending at least partway through the anchor, with a number of bores extending from an outer surface of the anchor to intersect the longitudinal opening. Adhesive (e.g., bone cement or osteogenic materials such as BMP) can be injected or otherwise introduced into the longitudinal opening and pass through the bores to at least partially fill portions of the canal surrounding the anchor. In various embodiments, an outer surface of some portion or all of the anchor can be porous or can include a plurality of depressions and/or other features for engaging with an adhesive within the canal.

In various embodiments, the design of the scapular anchor can be intended to engage or otherwise contact relatively hard cortical bone (or other anatomical structures) at one or more inner margins of the scapular canal. Such engagement with surrounding structures can desirably increase the ability of the anchor to remain secured within the canal under varying loading conditions of the anchor and/or glenoid tray, and the use of imaging data and/or computerized modeling as described herein can lead to the accurate and repeatable engineering of the scapula anchor and associated canal creation tools, as well as associated glenoid components and guide tools.

If desired, once the scapular anchor has been implanted and fixed in a desired location, and after the humeral stem has been implanted and fixed in a desired location (or where a trial scapular anchor and/or humeral stem have been implanted, respectively, or combinations thereof), a guide tool, jig or other measurement device can be employed or utilized to determine and/or measure the relationship between the scapular anchor and the humeral stem (either statically and/or dynamically), with the resulting measurements used to determine appropriate combinations of implant components that can be used to optimize the resulting surgical repair. For example, the measurement of the anchor and stem may indicate a need for an increased depth of the glenoid socket component, which may be accommodated by a glenoid “insert” having increased thicknesses at its peripheral walls (and/or an increased depth in the center of the insert cavity). Similarly, differing measurements may indicate a desire and/or need for differing humeral head designs and/or stem interfaces, as well as differing designs, angulations and/or shapes of glenoid implant components and/or glenoid inserts, which may be provided in multiple sizes and/or shapes including some patient-specific and/or patient-adapted features and other standard feature variations. In various exemplary embodiments, a glenoid implant insert could include a variety of inserts of differing thicknesses, including eccentric thickness that may alter the orientation and/or angulation of the resulting glenoid articulating surface(s) relative to the scapula and/or humerus. Similarly, a variety of inserts could include differing diameters and/or depths of the joint-facing concave surface as well as alterations and/or variations to the implant/surface rotational alignment relative to the glenoid axis, the flexion/extension angle and the version/retroversion angle. In various embodiments, the glenoid tray could include a first insert that establishes a desired glenoid articulating surface, and a second insert that establishes a desired glenoid rim geometry and/or thickness (e.g., a labrum replacement insert), with the two inserts connecting to the tray and/or each other in various arrangements.

In various other embodiments, once a glenoid tray is fixed to the scapula and secured to the scapular anchor, and a humeral head is secured to the humeral stem (or where a trial glenoid tray and/or humeral head have been positioned or otherwise implanted, respectively, or combinations thereof), various spacer and/or sizing tools could be employed to determine an appropriate size and/or shape of the glenoid insert (in a manner similar to a tibial insert and/or sizing template of a knee joint replacement procedure). In various embodiments, the spacer and/or sizing tools could allow and/or facilitate motion of the shoulder joint by the surgeon to assess joint tension and/or laxity, as well as kinematic movement of the surgical repair and implant components. Once a desired size and/or shape of the insert has been determined, the insert can be “docked,” implanted or otherwise secured within the glenoid tray, and the relevant soft tissue structures and surgical incision repaired and/or closed, in a typical manner.

At the end of a case, all relevant anatomical and alignment information can be saved for the patient file. This can be of great assistance to the surgeon in the future, including for use in planning of future surgeries, as well as to facilitate assessment of the shoulder during post-operative recovery, as the outcome of implant positioning can be seen and assessed before the formation of significant scar tissues and/or additional anatomical or implant structural degradation that may occur.

If desired, spacers, inserts or other measuring tools may be used to determine an appropriate size and/or shape of a glenoid tray insert (or other implant component). The spacers may correspond to one or more in a series of prosthetic humeral heads and/or a series of glenoid inserts (and/or combinations thereof). In use, the spacer can be pushed into the joint, between the glenoid tray and the humeral head, with progressively larger spacers employed in a known manner until a desired distraction, tension and/or other separation between the two components occurs. This assessment could include static as well as dynamic/kinematic measurements of the shoulder joint (e.g., measurements of one or a plurality of implant/shoulder orientations and/or positions, including still and/or range of motion measurements), and a desired humeral head and/or desired insert size/shape can be selected and implanted into the joint. In one exemplary embodiment, the physician can choose a desired humeral head size and/or orientation corresponding to a desired and/or proper articulation of the shoulder joint. Once the proper head size is determined, the prosthetic head can be permanently coupled to the stem. Once the head is positioned, impact forces can be imparted onto the head along a desired central axis, thereby coupling the head to the stem. In various alternative embodiments, the articulating or joint-facing surface of the glenoid prosthesis (which accommodates the head or prosthetic ball of the humerus) could be relatively smooth.

Where an opening is provided in the glenoid tray, a plug of suitable material, e.g., bone cement, metal, or other suitable materials such as plastic, can be provided in the opening to maintain a smooth surface, or a portion of the insert can include a feature that mates with the opening and secures the insert within the glenoid tray component. In various embodiments, the insert may comprise a wearing surface that is secured to the joint-facing surface of the tray, and it can be fastened within the tray by a variety of fastening techniques known for use in arthroplasty procedures, including adhesives, screws, detents, pins, and the like. If desired, the insert and/or humeral head may be designed for replacement after sufficient wear (e.g., after 15 or 20 years of continuous use by the patient). Of course, the various component features and fixation systems may be fabricated (e.g., by casting) as a single unitary construct (e.g., a unitary glenoid or humeral prosthesis and associated anchor/stem) using patient-specific and/or patient-adapted models, which may obviate or reduce the need for various modular embodiments and/or connection schemes illustrated and described herein.

Following implantation, the soft tissue balance and/or other kinematics of the shoulder joint can again be assessed, if desired, and then the split in the rotator interval can be closed. The deltoid can be repaired back to the acromion. Subcutaneous tissues and skin can then be closed per the surgeon's usual routine.

If trial components are used, the surgeon can assess alignment and stability of the trial components and the joint. During this assessment, the surgeon may conduct certain assessment processes such as external/internal rotation, rotary laxity testing, range of motion testing (external rotation, internal rotation and elevation) and stability testing (anterior, posterior and inferior translation). Thus, in an external/internal rotation test, the surgeon can position the humerus at the first location and visualize the shoulder directly (e.g., visually and/or via endoscopic optics) and/or by utilizing non-invasive imaging system such as a fluoroscope (e.g., activated by depressing a foot pedal actuator). If desired, the surgeon can then position the humerus at a second location and once again visualize the shoulder directly (e.g., visually and/or via endoscopic optics) and/or by utilizing non-invasive imaging system such as a fluoroscope (e.g., by depressing a foot pedal actuator). If desired, a computing system can register and/or store the respective location data for display and/or calculation of rotation/kinematics for the surgeon and/or automated system to determine whether the data is acceptable for the patient and the product involved. If not, the computer can apply rules in order to generate and display suggestions for releasing ligaments or other tissue, or using other component sizes or types. Once the proper tissue releases have been made, if necessary, and alignment and stability are acceptable as noted quantitatively on screen about all axes, the relevant trial components may be removed and actual components installed, and assessed in performance in a manner similar to that in which the trial components were installed, and assessed.

In alternative embodiments, the above-described assessment process can be utilized with the actual implant components installed, as opposed to trial components, as desired.

Depending upon the type, location and orientation of the surgical access path(s), as well as the features and specific of the relevant anatomical structures, various alternative embodiments of one or more sets of jigs can be designed to facilitate and accommodate surgical procedures in the shoulder. Desirably, the jigs can be designed and/or selected in connection with the design and/or selection of a patient-specific and patient-adapted implant component. The various jig designs desirably guide the surgeon in performing one or more patient-specific cuts or other surgical steps to the bone or other tissues so that the cut bone surface(s) negatively-match or otherwise accommodate corresponding surfaces (such as patient-specific bone cuts-facing surfaces) of the implant component. In various embodiments, alternative jig sets can be designed and supplied to facilitate one or more alternative surgical approaches, such as individual superior and anterior approaches, allowing a surgeon to choose a desired surgical approach option during the surgery.

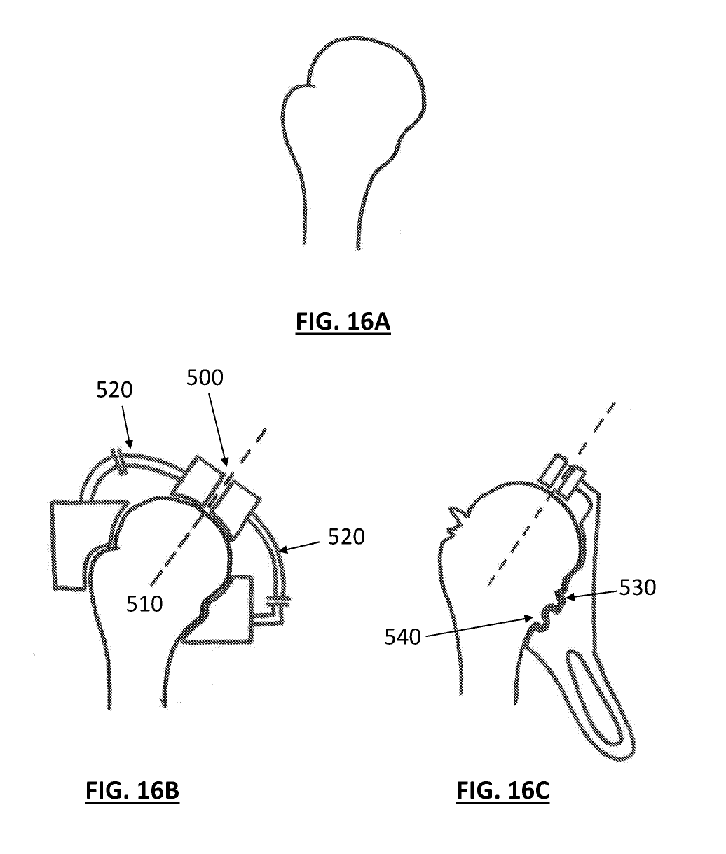

FIG. 16A depicts a normal humeral head and upper humerus which forms part of a shoulder joint. FIG. 16B depicts the humeral head of FIG. 16A with an alignment jig or guide tool designed to identify and locate various portions of the humeral anatomy. In this embodiment, a jig having a plurality of conforming surfaces has been designed using patient-specific information regarding one or more of the humerus, the humeral neck, the greater tuberosity and/or the lesser tuberosity of the humerus. Desirably, the conforming surfaces will fit onto the humerus on only one position and orientation, thereby aligning the jig relative to the humerus in a known position. This embodiment desirably incorporates an alignment hole 500 which aligns with an axis 510 of the humeral head. After proper positioning of the jig, a pin or other mechanism (e.g., drill, reamer, etc.) can be inserted into the hole 500, and provide a secure reference point for various surgical operations, including the reaming of the humeral head and/or drilling of the axis 510 in preparation for a humeral head resurfacing implant or other surgical procedure. The alignment mechanisms may be connected to the one or more conforming surfaces by linkages 520 (removable, moveable and/or fixed) or other devices, or the entire jig may be formed from a single piece and extend over a substantial portion and/or unique features of the humeral head and/or other bone.

FIG. 16C depicts an alternative embodiment of a humeral head jig that utilizes a single conforming surface 530 to align the jig. In this embodiment, one or more protrusions or osteophytes 540 is mirrored by the conforming surfaces, which permits alignment and positioning of the jig in a known manner.

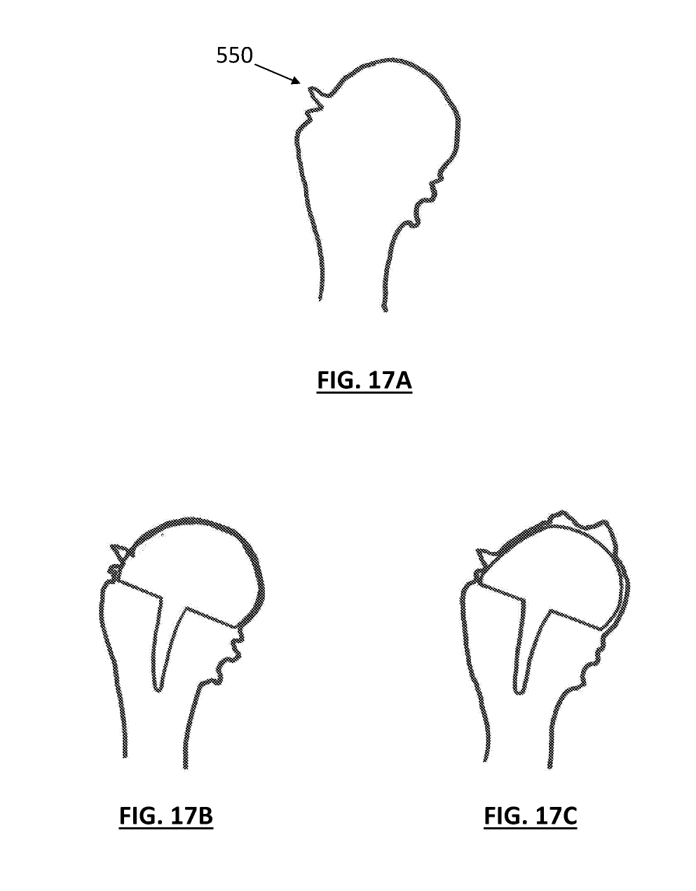

FIG. 17A depicts a humeral head with osteophytes 550, and FIGS. 17B and 17C depict the humeral head with a more normalized surface that has been corrected by virtual removal of the osteophytes.

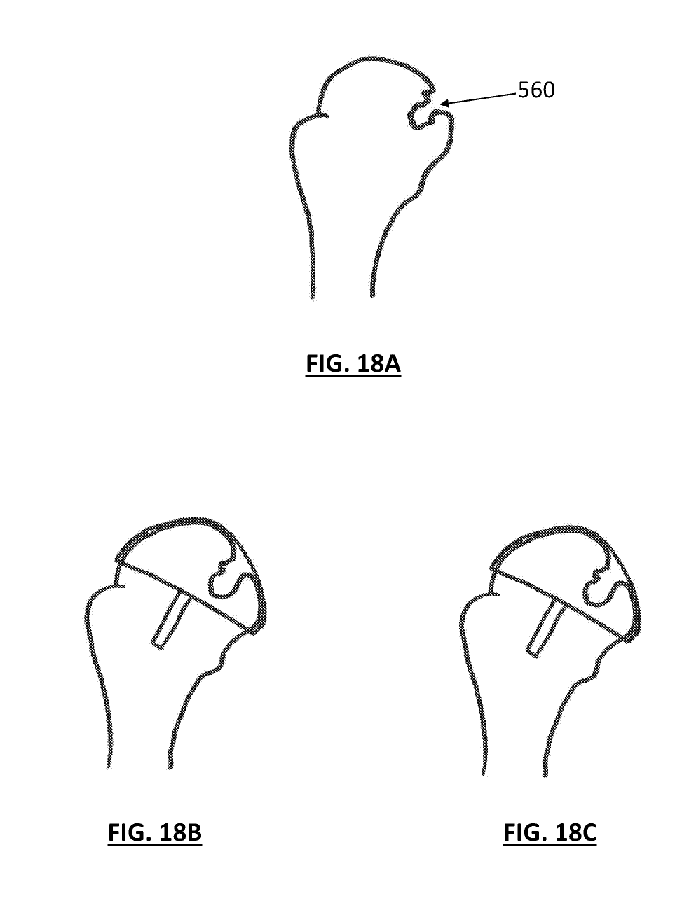

FIG. 18A depicts a humeral head with voids, fissures or cysts 560, and FIGS. 18B and 18C depict the humeral head with a more normalized surface that has been corrected by virtual removal of the voids, fissures or cysts.

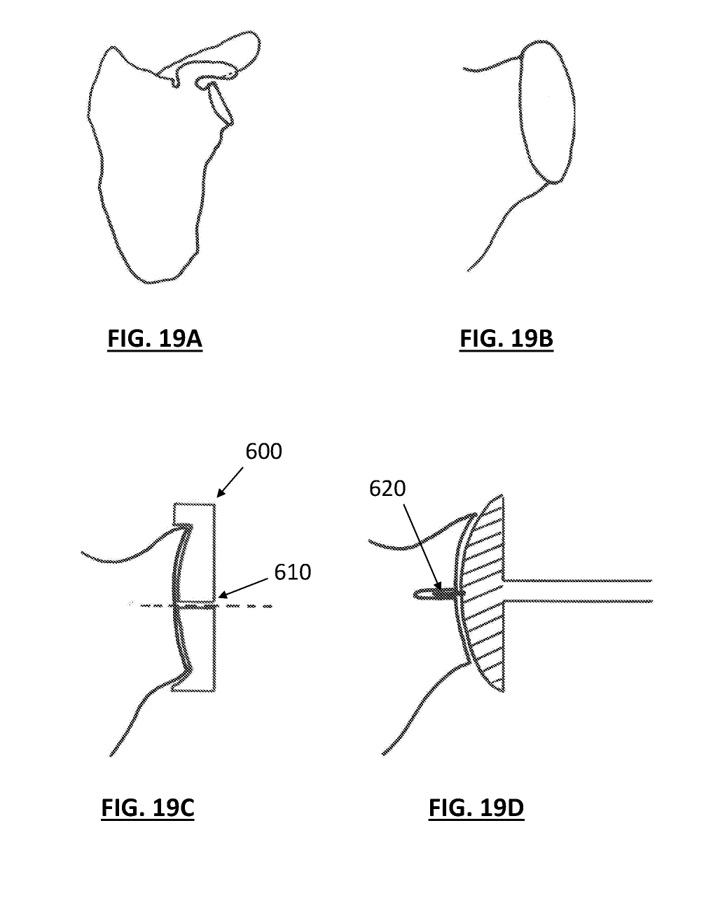

FIG. 19A depicts a healthy scapula of a shoulder joint, FIG. 19B depicts a normal glenoid component of the shoulder of FIG. 19A, and FIG. 19C depicts one embodiment of an alignment jig 600 for use in preparing the relevant anatomical features of the glenoid and/or scapula for an implant component. As previously described in connection with various other embodiments, the jig 600 may comprise one or more conforming surfaces that are shaped to mirror the patient-specific anatomy of the glenoid, allowing the jig to be positioned on the glenoid in a known position and orientation. An alignment hole 610 in the glenoid jig provides a desired pathway for orienting and inserting a pin 620 or other alignment mechanism, or to provide a pathway for a drilling or reaming device. After the pin 620 has been inserted, the jig 600 can be removed and the pin 620 utilized as a secure reference point for various surgical operations, including the milling and/or reaming of the glenoid in preparation for a glenoid component of a shoulder joint replacement/resurfacing implant (see FIG. 19D).

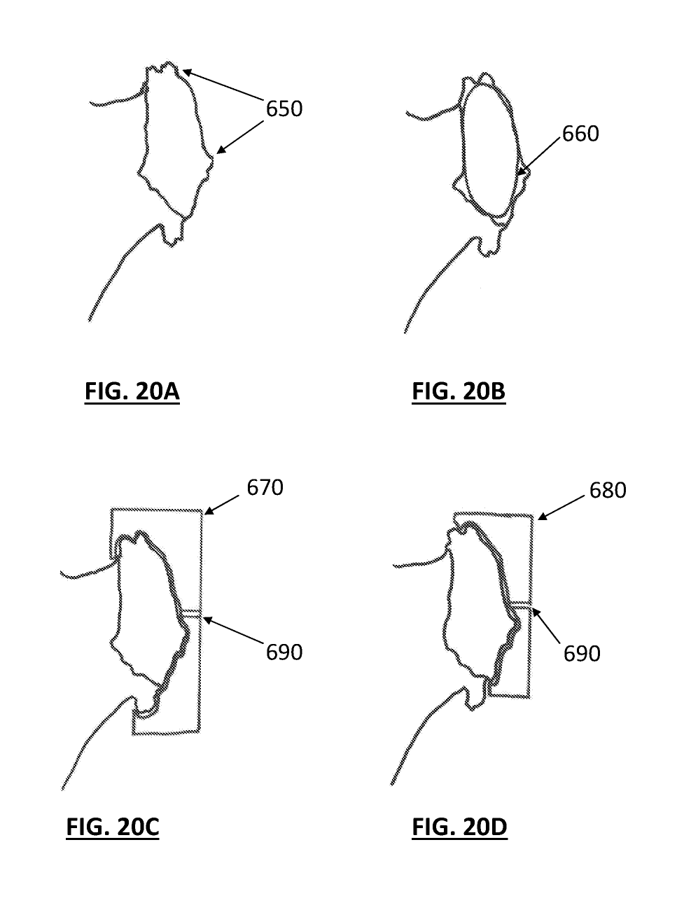

FIG. 20A depicts a glenoid surface with osteophytes 650, and FIG. 20B depicts the glenoid surface with a more normalized surface 660 that has been corrected by virtual removal of the osteophytes. FIGS. 20C and 20D depict two alternative embodiments of glenoid jigs 670 and 680 for use in preparing the glenoid surface, with each of the jigs 670 and 680 incorporating conforming surfaces (as previously described) that accommodate the osteophytes. If desired, the jig of FIG. 20C can be formed from an elastic or flexible material to allow it to “snap fit” over the glenoid surface and associated osteophytes. As previously noted, the jigs 670 and 680 can include various alignment holes 690 or slots, etc., to facilitate, guide and/or otherwise allow placement of pins or other surgical actions (not shown).

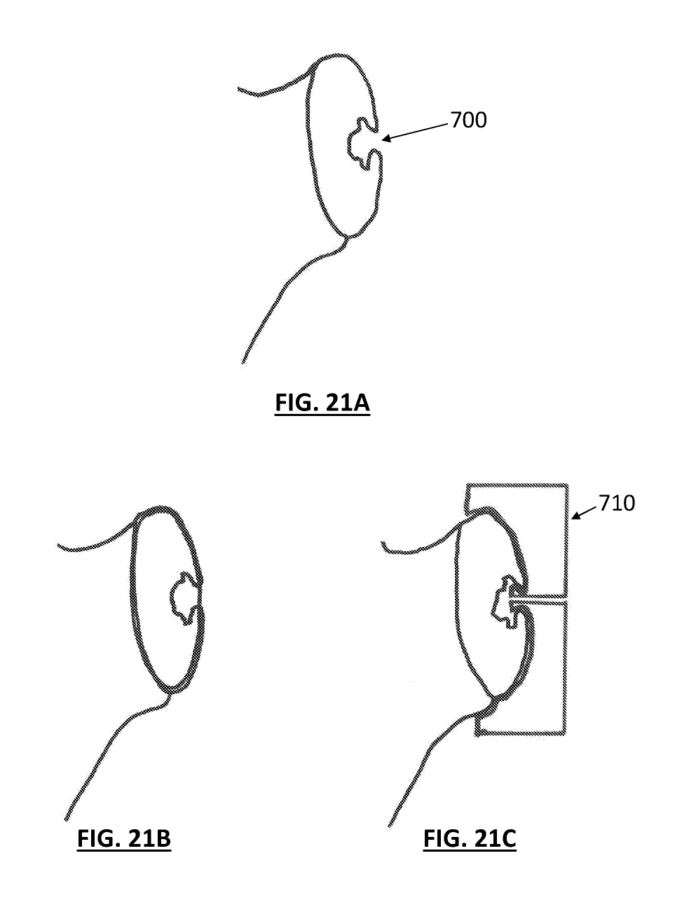

FIG. 21A depicts a glenoid surface with voids, fissures or cysts 700, and FIG. 21B depicts the glenoid surface with a more normalized surface that has been corrected by virtual “filling” of the voids, fissures or cysts. FIG. 21C depicts one embodiment of a glenoid jig 710 for use in preparing the glenoid surface, with the jig 710 incorporating various conforming surfaces that accommodate the voids, fissures and/or cysts (and/or other surfaces) of the glenoid surface.

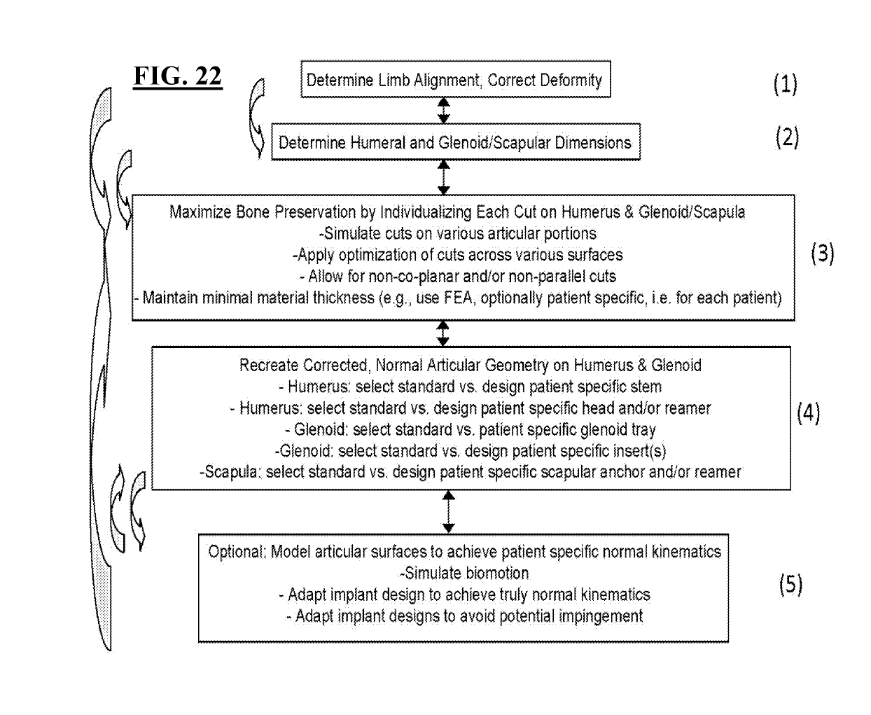

FIG. 22 shows an exemplary flowchart of a process beginning with the collection of patient data in process steps. This data is used by process to convert and display the native anatomy to a user. In various process steps, the image data can be used with implant specific data to design guide tools and/or other instruments. The exemplary process shown in FIG. 22 includes four general steps and, optionally, can include a fifth general step. Each general step includes various specific steps. The general steps are identified as (1)-(5) in the figure. These steps can be performed virtually, for example, by using one or more computers that have or can receive patient-specific data and specifically configured software or instructions to perform such steps.

In general step (1), limb alignment and deformity corrections are determined, to the extent that either is needed for a specific patient's situation. In general step (2), the requisite humeral and glenoid/scapular dimensions of the implant components are determined based on patient-specific data obtained, for example, from image data of the patient's shoulder.

In general step (3), bone preservation is maximized by virtually determining a resection cut strategy for the patient's humerus and glenoid/scapula that provides minimal bone loss optionally while also meeting other user-defined parameters such as, for example, maintaining a minimum implant thickness, using certain resection cuts to help correct the patient's misalignment, removing diseased or undesired portions of the patient's bone or anatomy, and/or other parameters. This general step can include one or more of the steps of (i) simulating resection cuts on one or both articular sides (e.g., on the humerus and/or glenoid), (ii) applying optimized cuts across one or both articular sides, (iii) allowing for non-co-planar and/or non-parallel resection cuts and (iv) maintaining and/or determining minimal material thickness. The minimal material thickness for the implant selection and/or design can be an established threshold, for example, as previously determined by a finite element analysis (“FEA”) of the implant's standard characteristics and features. Alternatively, the minimal material thickness can be determined for the specific implant, for example, as determined by an FEA of the implant's standard and patient-specific characteristics and features. If desired, FEA and/or other load-bearing/modeling analysis may be used to further optimize or otherwise modify the individual implant design, such as where the implant is under or over-engineered than required to accommodate the patient's biomechanical needs, or is otherwise undesirable in one or more aspects relative to such analysis. In such a case, the implant design may be further modified and/or redesigned to more accurately accommodate the patient's needs, which may have the side effect of increasing/reducing implant characteristics (e.g., size, shape or thickness) or otherwise modifying one or more of the various design “constraints” or limitations currently accommodated by the present design features of the implant. If desired, this step can also assist in identifying for a surgeon the bone resection design to perform in the surgical theater and it also identifies the design of the bone-facing surface(s) of the implant components, which substantially negatively-match the patient's resected bone surfaces, at least in part.

In general step (4), a corrected, normal and/or optimized articular geometry on the humerus and glenoid is recreated virtually. For the humerus, this general step can include, for example, the step of: (i) selecting a standard or selecting and/or designing a patient-engineered or patient-specific stem; and (ii) selecting a standard or selecting and/or designing a patient-specific or patient-engineered head and/or reamer (or other surgical tools). If desired, the humeral head and the glenoid surface(s) can include the same, similar or different curvatures. For the glenoid, this general step includes the step of selecting a standard or selecting and/or designing a patient-specific or patient-engineered glenoid tray, as well as the step of selecting a standard insert articular surface(s) or selecting and/or designing a patient-specific or patient-engineered articular surface(s). For the scapular anchor, this general step can include the step of selecting a standard or selecting and/or designing a patient-specific or patient-engineered scapular anchor, reamer and/or other tools.

In various embodiments, the insert(s) can include patient-specific poly-articular surface(s) selected and/or designed, for example, to simulate the normal or optimized three-dimensional geometry of the patient's tibial articular surface and/or surrounding periphery. The patient-engineered poly-articular surface can be selected and/or designed, for example, to optimize kinematics with the bearing surfaces of the humeral implant component. This step can be used to define the bearing portion of the outer, joint-facing surfaces (e.g., articular surfaces) of the implant components.

In optional general step (5), a virtual implant model (for example, generated and displayed using a computer specifically configured with software and/or instructions to assess and display such models) is assessed and can be altered to achieve normal or optimized kinematics for the patient. For example, the outer joint-facing or articular surface(s) of one or more implant components can be assessed and adapted to improve kinematics for the patient. This general step can include one or more of the steps of: (i) virtually simulating biomotion of the model, (ii) adapting the implant design to achieve normal or optimized kinematics for the patient, and (iii) adapting the implant design to avoid potential impingement.

In one exemplary embodiment, the following modeling and derivation steps can be utilized to create a desired implant design, as well as be used to estimate or derive a shape or curvature, wherein the shape or curvature information can be improved by combining it with information about other anatomic features and/or design, availability, cost or other constraints for the implant:

(1) construct outer cartilage surface from edges of multiple faceted cuts;

(2) define multiple virtual bone cuts, extract various curvatures, apply best fit analysis for closest implant, adapt best fit on various anatomical and modeled measurements;

(3) apply predefined virtual bone cuts according to design rules (best fit, bone preservation, minimum required supporting bone structures, etc.), if any;

(4) select implant; and

(5) optionally reduce or otherwise alter number of cuts after surface has been constructed to obtain a desired number of cut inner surfaces.

In various alternative embodiments, the humeral and glenoid/scapular bones of the anatomy can be initially resected and/or prepared, and then the various implant components (including any stems and/or anchors, if not already implanted) can be implanted. During insertion of the various components, it may become apparent that one or more bones may need to be further prepared, such as broaching the intra-medullary (IM) canal of the humerus that is not sufficiently prepared for a given stem. In such embodiments, additional surgical tools may be provided and used to broach a selected portion of the IM canal of the humerus. Various sizes of broaches (including standard as well as patient-adapted and/or patient-specific broaches) may be used to progressively enlarge the broached area of the humerus.

After inserting a humeral stem into the medullary canal using impaction, a humeral head can be coupled to a locking taper (or other fixation mechanism) formed on the stem proximal end. A similar arrangement can be employed with the glenoid tray and scapular anchor, if desired. The various coupling mechanisms can be aligned within the patient to place a stem/anchor axis in alignment with the attached head/tray, facilitating the use of an impact force applied to the head/tray in alignment with a direction of the coupling mechanism and/or axis of the stem/anchor.

In various alternative embodiments, the use of a reverse shoulder prosthesis is contemplated with appropriate variations in the described procedure. If desired, a similar superior approach can be used to implant the reverse shoulder prosthetic, which can include a cup member at a proximal end of the humeral stem and a spherical glenoid implant positioned at a resected glenoid. It is envisioned the cup member and glenoid implant can include fixation members (e.g., humeral stems and/or scapular anchors) as previously described. In one such embodiment of a reverse total shoulder arthroplasty, the glenoid component may approximate 5 degrees of inferior inclination, close to neutral version, and slight inferior translation to minimize notching. Such a design will desirably reference the inclination and version of the glenoid component from the sagittal plane, as previously defined and described. For example, the inclination plane could pass through an axis created by the intersection of the sagittal and transverse planes at 4 degrees of superior inclination. A second axis could then pass through the coronal and inclination plane. The version plane could pass through said second axis at 1 degree of retroversion. Such a design could allow the version plane to represent the proper orientation of the glenoid component—the glenoid component plane. The system could further include a glenoid guided tool used to target peripheral fixation screws and/or scapular anchors for the glenoid component. After pre-operatively determining the depth of the reaming operation used to seat the glenoid component, the surgeon or engineer could pre-operatively determine the number, length, and alignment of said peripheral fixation screws, which could include multiple screws at differing orientations (e.g., some screws angled relatively downwards, and others angled relatively upwards) as well as screws having directions opposed or otherwise not aligned with a primary longitudinal axis of the scapular anchor. The guide tool could have a mating surface that is the 3D inverse of the reamed surface. The guide tool could include a center hole in line with the scapular anchor and/or any central peg hole. In addition, peripheral holes in the guide tool could be in line with the pre-operatively planned screw locations. Drill taps could be passed through the peripheral holes. The guide tool could also have one or more marks or other indicators on a visible surface (e.g. a mark of the lateral surface pointing superiorly) to aid in the rotational alignment of the guide tool. During surgery, the surgeon could use an electrocautery instrument (or other instrument) to mark the surface of the glenoid (e.g. a mark pointing superiorly). The instrument's mark could eventually be aligned to the glenoid's surface mark, which could potentially be visualized through slots or other openings on a subsequent instrument and/or implant component to verify the seating and proper orientation of the instrument on the reamed bone. With regards to the humeral component, the position of the component could approximate a neutral retroversion, if desired.

In various embodiments, the design, selection and/or optimization of implant components and surgical procedures can include an automated analysis of the strength, durability and fatigue resistance of implant components as well as the bones in which they are to be implanted. In addition to optimizing bone preservation, including the maximum retention of anatomical support structures in critical areas such as the scapula, another factor in determining the depth, number, and/or orientation of resection cuts and/or implant component bone cuts is desired implant thickness. A minimum implant thickness can be included as part of the resection cut and/or bone cut design to ensure a threshold strength for the implant in the face of the stresses and forces associated with joint motion, such as lifting, hanging and pushing/pulling. In various embodiments, a Finite Element Analysis (FEA) assessment may be conducted for implant components of various sizes and with various bone cut numbers and orientations. If desired, a similar analysis may be performed for the intended anatomical support structures (e.g., the glenoid/scapula and/or femur of the shoulder). Such analyses may indicate maximum principal stresses observed in FEA analysis that can be used to establish an acceptable minimum implant thickness for an implant component having a particular size and, optionally, for a particular patient (e.g., having a particular weight, age, activity level, etc). These results may indicate suboptimal designs for implants and/or surgical resection procedures, which may necessitate alterations to the intended procedure and/or implant component design in various manners. In this way, the threshold implant thickness, design and/or any implant component feature, as well as the intended bone resection, can be adapted to a particular patient based on a combination of patient-specific geometric data and on patient-specific anthropometric data.

In various embodiments, a visible or tactile mark, orientation or indication feature can be, for example, an etching or other marking that can be aligned to point to the bicipital groove. In other embodiments, the visible or tactile orientation feature could be a small protuberance or tab extending from the cap toward the bicipital groove or received at least in part into the bicipital groove to align and position the guide tool quickly and correctly. The tab could be sized and shaped to be fit into a corresponding portion of the bicipital groove.

In designing and/or selecting the various implant components features as described herein, the process can include generating and/or using a model, for example, a virtual model, of the patient's joint that includes the selected measurements and virtually fitting one or more selected and/or designed implants into the virtual model. This approach would desirably allow for iterative selection and/or design improvement and could include steps to virtually assess the fit, such as virtual kinematics assessment.

In various embodiments, the process of selecting an implant component also includes selecting one or more component features that optimizes the fit with another implant component. In particular, for an implant that includes a first implant component and a second implant component that engage, for example, at a joint interface, selection of the second implant component can include selecting a component having a surface that provides a best or desired fit to the engaging surface of the first implant component. For example, for a shoulder implant that may include a humeral implant component and a glenoid implant component, with one or both of components selected based, at least in part, on the fit of the outer, joint-facing surface with the outer-joint-facing surface of the other component. The fit assessment can include, for example, selecting the humeral head component and/or the glenoid tray and/or tray insert component that substantially negatively-matches the fit or optimizes engagement in one or more dimensions, for example, in the coronal and/or sagittal dimensions. For example, a surface shape of a non-metallic component that best matches the dimensions and shape of an opposing metallic or ceramic or other hard material suitable for an implant component. By performing this component matching, component wear can be reduced.

For example, if a metal backed glenoid tray component is used with one or more polyethylene inserts or if an all polyethylene glenoid implant component is used, the polyethylene may have a curved portion typically designed to mate with the humeral head in a low friction form. This mating can be optimized by selecting a polyethylene insert that is optimized or achieves an optimal fit with regard to one or more of: depth of the concavity, width of the concavity, length of the concavity and/or radius or radii of curvature of the concavity. A glenoid insert and opposing humeral head surface can have can have a single or a composite radius of curvature in one or more dimensions, e.g., the coronal plane. They can also have multiple radii of curvature. Similar matching of polyethylene or other plastic shape to opposing metal or ceramic component shape can be performed in other joints.

Those of skill in the art will appreciate that a combination of standard and customized components may be used in conjunction with each other. For example, a standard tray component may be used with an insert component that has been individually constructed for a specific patient based on the patient's anatomy and joint information. Various embodiments incorporate a glenoid tray component with an insert component shaped so that once combined, they create a uniformly shaped implant matching the geometries of the patient's specific joint.

In various embodiments, a glenoid component (metal backed, ceramic or all plastic, e.g. polyethylene, or any other known in the art or developed in the future) can be designed or selected or adapted so that its peripheral margin will be closely matched to the patient specific glenoid rim or perimeter. Optionally, reaming can be simulated for placement of a glenoid component and the implant can then be designed or selected or adapted so that it will be closely matched to the resultant glenoid rim after reaming or other bone removal. Thus, the exterior dimensions of the implant, e.g. the rim and/or curvature(s) can be matched to the patient's geometry in this fashion. Curvatures of the exterior, bone facing shape of the glenoid component can have constant or variable radii in one, two or three dimensions. At least one or more of these curvatures or surfaces can be adapted to the patient's shape in one or more dimensions, optionally adapted to the result of a simulated surgical alteration of the anatomy, e.g. reaming, the removal of osteophytes or cutting. For example, if a cut is performed, the implant can be adapted to the perimeter of the bone resulting after the cut has been placed. In this setting, at least a portion of the perimeter of the implant can be adapted to the perimeter of the patient's cut bone. The undersurface of the implant can then be flat, facing the cut bone, or conical in shape. The glenoid component can be selected, adapted or designed to rest on the glenoid rim or extend beyond the glenoid rim, resting on portions of cortical bone or, for example, also osteophytes. In this embodiment, the glenoid fossa facing portion of the component can have standard dimensions, e.g. approximating those of a reamer used for reaming the glenoid fossa, while the peripheral portions, e.g. those facing the glenoid rim or cortical bone, e.g. on the anterior or posterior aspect of the scapula, can be patient specific or patient adapted. Any of these embodiments can be applicable to shoulder resurfacing techniques and implants as well as shoulder replacement techniques and implants, including primary and revision shoulder systems, as well as reverse or inverse shoulder systems.

If desired, the patient-specific data can be utilized to create a reaming guide or other tools for preparing the glenoid for an implant component. To avoid cutting/reaming through a glenoid in a reaming operation, it may be desirous to have a guide or other tool arrangement or design that limits reamer motion or movement in various manners to one or more predetermined depths that were previously determined using patient-specific data, e.g. pre-operative CT or MRI or intraoperative ultrasound measurement of glenoid depths. Such a tool can comprise a patient-matched surface on the glenoid and/or other anatomical structures. Desirably, the tool can control both placement and depth of reaming tools to a desired degree. Moreover, the planning and design phase of such a guide tool can potentially identify any “at risk” operations for patients especially susceptible to such dangers, and possibly the implant design can be redesigned to accommodate the special needs of such patients as well.

Optionally, standard, round dimensions of a polyethylene or other inserts can be used with various embodiments described herein.

Similarly, a glenoid component can be selected for, adapted to or matched to the glenoid rim, optionally after surgically preparing or resectioning all or portions of the glenoid rim including osteophytes.

In various embodiments, a metal backed or ceramic glenoid component can include external, bone facing patient specific features and shapes, while the internal, insert facing shape can be standard. For example, a standard polyethylene insert can be locked into a patient specific glenoid component; the glenoid component having patient specific features or shapes on the external, bone facing side, while the internal dimensions or shape can be standard. The external bone facing patient specific features and shape can help achieve a desired implant orientation and/or position including a desired anteversion or retroversion. The internal dimensions can be standard and can be designed with a locking feature to hold a standard insert in place. The standard insert locked into the glenoid metal backed or ceramic component can have a smooth flat or concave bearing surface to articulate with a humeral head component. The humeral head component can, optionally, be modular in design. The humeral component can be selected for a patient, adapted to a patient or designed for a patient using an imaging test. The imaging test can be used to select or adapt or design a shape with any one of the following geometries matched, adapted to or selected for the patient using the one or more scan data:
Component thickness
Component diameter
Entry angle into the humeral shaft
Humeral neck angle
Stem curvature

Optionally, a resurfacing humeral head component can be used with at least portions of a bone facing surface selected for, adapted to or designed for aspects of the patient's humeral head shape.

Any joint implant components, including those for a shoulder or other joint, can be formed or adapted based on a pre-existing blank. For example in a shoulder joint (but also in any other joint or a spine), an imaging test, e.g., a CT or MRI, can be obtained to generate information, for example, about the shape or dimensions of the humerus or the glenoid, as well as any other portions of the joint. Various dimensions or shapes of the joint can be determined and a pre-existing blank humerus or glenoid component can then be selected. The shape of the pre-existing blank humerus or glenoid component can then be adapted to the patient's shape, for example, by selectively removing material, e.g. with a machining or cutting or abrasion or other process, or by adding material. The shape of the blank will generally be selected to be smaller than the target anatomy when material is added to achieve the patient adapted or patient specific implant features or surfaces. The shape of the blank will generally be selected to be larger than the target anatomy when material is removed to achieve the patient adapted or patient specific implant features or surfaces. Any manufacturing process known in the art or developed in the future can be used to add or remove material, including for metals, ceramics, plastics and other materials.

An outer, bone facing component can be adapted to or matched to the patient's anatomic features using a blank in this manner. Alternatively or additionally, an insert can be adapted or shaped based on the patient's anatomic features in one or two or three dimensions. For example, a standard insert, e.g. with a standard locking mechanism into the outer component, can be adapted so that its outer rim will not overhang the patient's anatomy, e.g. a glenoid rim, before or after a surgical alteration such as a cutting or reaming. The surgical alteration can, in this example as well as in many of the foregoing and following embodiments, be simulated on a computer and the insert blank can then be shaped based on the result of the simulation. Thus, a glenoid insert as well as a metal backing can be adapted, e.g. machined, so that its perimeter will match the glenoid rim in at least a portion either before or after the surgical alteration of the glenoid.

Implant components can be attached to the underlying bone. Any attachment mechanism known in the art can be used, e.g. pegs, fins, keels, stems, anchors, pins and the like. The attachment mechanisms can be standard in at least one of shape, size and location. Thus, in a glenoid component, an all polyethylene component can be used. Using imaging data, the blank glenoid component can be aligned relative to the patient's glenoid (optionally after a simulated surgical intervention) to optimize the position of any standard attachment mechanisms relative to the bone to which they are intended to be attached. Once the optimal position of the glenoid blank and its attachment mechanisms has been selected, the outer rim and, optionally, the bearing surface of the component can be adapted based on the patient's anatomy. Thus, for example, the outer periphery of the implant can be machined then to substantially align with portions of the patient's glenoid rim.

Alternatively, rather than using standard attachment mechanisms, the position and orientation of any peg, keel or other fixation features of glenoid components or implant components in any other joint can be designed, adapted, shaped, changed or optimized relative to the patient's geometry, e.g. relative to the adjacent cortex or, for example, the center of a medullary cavity or other anatomic or geometric features. In a glenoid, the length and width of the attachment mechanisms can be adapted to the mediolateral width of the glenoid or to the existing bone stock available or any other glenoid dimension, e.g. superoinferior.

The articular surface of a glenoid component can have a standard geometry in one or more dimensions or can be completely standard. The articular surface of the glenoid component can also include patient specific or patient derived shapes. For example, the articular surface of the glenoid component can be derived using the curvature or shape of the cartilage or subchondral bone of the patient, on the glenoid or the humeral side, in one or more dimensions or directions. Alternatively, the articular surface of a humeral component can be derived using the curvature or shape of the cartilage or subchondral bone of the patient on the humerus or glenoid in one or more dimensions or directions and the articular surface of the glenoid component can be selected or adapted or designed based on the humeral component implant shape. The selection, adaption or design can occur using a set of rules, e.g. desirable humeral to glenoid articular surface radius ratios, in one or more planes, e.g. superoinferior or mediolateral.

In various embodiments, the thickness of one or more implant components or portions of one or more implant components can be selected or adapted or designed based on one or more geometric features of a patient or patient weight or height or BMI or other patient specific characteristics, e.g. gender, lifestyle, activity level etc. This selection or adaptation or design can be performed for any implant component in a shoulder or other human joint. For example, in a shoulder, a glenoid component thickness can be selected, adapted or designed based on one or more of a patient's humeral or glenoid AP or ML or SI dimensions, humeral or glenoid sagittal curvature, humeral or glenoid coronal curvature, estimated contact area, estimated contact stresses, biomechanical loads, optionally for different flexion and extension angles, glenoid bone stock and the like. The metal, ceramic or plastic thickness as well as the thickness of one or more optional inserts can be selected, adapted or designed using this or similar information.

Various portions and embodiments described herein can be provided in a kit, which can include various combinations of patient-specific and/or patient-adapted implant and/or tools, including glenoid and/or humeral implant components, guide tools, jigs, and surgical instruments such as saws, drills and broaches. Various components, tools and/or procedural steps can include standard features alone and/or in combination with patient-specific and/or patient-adapted features. If desired, various portions of the kit can be used for a plurality of procedures and need not be customized for a particular procedure or patient. Further, the kit can include a plurality of portions that allow it to be used in several procedures for many differing anatomies, sizes, and the like. Further, various other portions, such as the reamers and/or other tools can be appropriate for a plurality of different patients.

The various techniques and devices described herein, as well as the image and modeling information provided by systems and processes according to the present disclosure, may facilitate telemedical techniques, because they provide useful images for distribution to distant geographic locations where expert surgical or medical specialists may collaborate during surgery. Thus, systems and processes according to the present disclosure can be used in connection with computing functionality which is networked or otherwise in communication with computing functionality in other locations, whether by PSTN, information exchange infrastructures such as packet switched networks including the Internet, or as otherwise desired. Such remote imaging may occur on computers, wireless devices, videoconferencing devices or in any other mode or on any other platform which is now or may in the future be capable of rending images or parts of them produced in accordance with the present disclosure. Parallel communication links such as switched or unswitched telephone call connections may also accompany or form part of such telemedical techniques. Distant databases such as online catalogs of implant suppliers or prosthetics buyers or distributors may form part of or be networked with computing functionality to give the surgeon in real time access to additional options for implants which could be procured and used during the surgical operation.

### Example

### Surgical Planning, Implant and Surgical Tool Design, Selection, and/or Adaptation

In an exemplary embodiment, image data on a patient's diseased or damaged shoulder joint is obtained, and the image data includes information about the patient's bone stock, particularly of the shoulder joint. Based on the image data (e.g., the glenoid shape of the patient's shoulder joint), an implant can be designed, selected, and/or adapted. Such design, selection and/or adaptation can optionally include patient-specific design of an anchoring mechanism, including pegs or anchors, and the patient-specific design may include patient-specific peg/anchor location, size, and/or shape.

Based on the patient's data or information, a surgical plan can be customized. For example, in view of the patient's bone stock, a surgical procedure (e.g., standard vs. reverse) may be selected. The surgical plan may also incorporate the surgeon's own preferences (e.g., anterior or posterior, or combined approach).

An implant may be designed, selected and/or adapted for the patient. Such an implant can include patient-specific information, including, e.g., the glenoid shape and size, and the bone stock. For example, the size, shape, and one or more dimensions of the implant can be adjusted in view of the patient's bone stock. The positioning of the implant (e.g., through one or more anchoring mechanisms) may also be adjusted relative to the bone stock.

One or more surgical tools can also be customized for the patient, e.g., based on the surgical plan, the patient's data or information (e.g., bone stock), and/or the implant.

The size, shape, position and/or orientation of the implant or the one or more surgical tools can be adjusted based on information about the patient's cortical bone (e.g., thickness), bone density, bone strength, bone quality, as well as biomechanical or kinematic properties.

It should be noted the steps described above can be iterative and in alternative orders, in order to optimize the surgical plan, the implant, and/or the surgical tools for a particular patient in accordance with a surgeon's particular preferences (including both general preferences and case-specific or patient-specific preferences).

The entire disclosure of each of the publications, patent documents, and other references referred to herein is incorporated herein by reference in its entirety for all purposes to the same extent as if each individual source were individually denoted as being incorporated by reference.

The various descriptions contained herein are merely exemplary in nature and, thus, variations that do not depart from the gist of the teachings are intended to be within the scope of the teachings. Such variations are not to be regarded as a departure from the spirit and scope of the teachings, and the mixing and matching of various features, elements and/or functions between various embodiments is expressly contemplated herein. One of ordinary skill in the art would appreciate from this disclosure that features, elements and/or functions of one embodiment may be incorporated into another embodiment as appropriate, unless described otherwise above. Many additional changes in the details, materials, and arrangement of parts, herein described and illustrated, can be made by those skilled in the art. Accordingly, it will be understood that the disclosure should not be limited to the embodiments disclosed herein, but can include practices otherwise than specifically described, and are to be interpreted as broadly as allowed under the law.

What is claimed is:

1. An implant system for treating a shoulder joint of a patient, the shoulder joint including a scapula, the scapula including a glenoid structure, the implant comprising:
a glenoid implant component, the glenoid implant component having a medial surface and a lateral surface, wherein the medial surface is configured to be coupled to a resected surface of the glenoid structure and has one or more anchoring protrusions, and wherein the lateral surface includes a curved portion configured to mate with a humeral head of a humeral implant component; and
a scapular anchor component, the scapular anchor component configured, based, at least in part, on patient-specific information, to extend from the medial surface of the glenoid implant component and into a canal in the lateral border of the scapula.

2. The implant system of claim 1, wherein the glenoid implant component includes an engagement structure configured to engage the scapular anchor component.

3. The implant system of claim 1, wherein the glenoid implant component and the scapular anchor comprise a one-piece implant.

4. The implant system of claim 1, wherein at least one characteristic of the scapular anchor component selected from the group of characteristics consisting of a length, a diameter, a shape, and combinations thereof corresponds to one or more of a length, a diameter, or a shape of at least a portion of the canal.

5. The implant system of claim 1, wherein an angle formed between the scapular anchor and the glenoid implant component corresponds to an angle between the canal and the glenoid structure of the shoulder joint.

6. A method of making the implant system of claim 1.
